(19) United States
(12) Patent Application Publication
(10) Pub. No.: US 2021/0307760 A1
(43) Pub. Date: Oct. 7, 2021

(54) Intrasacular Aneurysm Occlusion Device with a Flexible Net or Mesh and Polymer Longitudinal Embolic Members

(71) Applicant: Robert A. Connor, St. Paul, MN (US)

(72) Inventor: Robert A. Connor, St. Paul, MN (US)

(73) Assignee: Aneuclose LLC, St. Paul, MN (US)

(21) Appl. No.: 17/353,652

(22) Filed: Jun. 21, 2021

Related U.S. Application Data
(63) Continuation-in-part of application No. 17/220,002, filed on Apr. 1, 2021.
Application No. 17/220,002 is a continuation-in-part of application No. 17/214,827, filed on Mar. 27, 2021.
Application No. 17/214,827 is a continuation-in-part of application No. 17/211,446, filed on Mar. 24, 2021.
Application No. 17/211,446 is a continuation-in-part of application No. 16/693,267, filed on Nov. 23, 2019.
Application No. 16/693,267 is a continuation-in-part of application No. 16/660,929, filed on Oct. 23, 2019.
Application No. 16/693,267 is a continuation-in-part of application No. 16/660,929, filed on Oct. 23, 2019.
Application No. 16/660,929 is a continuation-in-part of application No. 16/541,241, filed on Aug. 15, 2019.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 16/541,241, filed on Aug. 15, 2019.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 15/865,822, filed on Jan. 9, 2018, now Pat. No. 10,716,573.
Application No. 15/865,822 is a continuation-in-part of application No. 15/081,909, filed on Mar. 27, 2016, now abandoned.
Application No. 15/081,909 is a continuation-in-part of application No. 14/526,600, filed on Oct. 29, 2014, now abandoned.
Application No. 14/526,600 is a continuation-in-part of application No. 15/080,915, filed on Mar. 25, 2016, now Pat. No. 10,028,747.
Application No. 15/080,915 is a continuation-in-part of application No. 14/526,600, filed on Oct. 29, 2014, now abandoned.
Application No. 15/081,909 is a continuation-in-part of application No. 14/526,600, filed on Oct. 29, 2014, now abandoned.
Application No. 15/080,915 is a continuation-in-part of application No. 14/526,600, filed on Oct. 29, 2014, now abandoned.
Application No. 14/526,600 is a continuation-in-part of application No. 12/989,048, filed on Oct. 21, 2010, now Pat. No. 8,974,487.
(60) Provisional application No. 63/119,774, filed on Dec. 1, 2020.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/720,173, filed on Aug. 21, 2018.
(60) Provisional application No. 62/589,754, filed on Nov. 22, 2017.
(60) Provisional application No. 62/472,519, filed on Mar. 16, 2017.
(60) Provisional application No. 62/589,754, filed on Nov. 22, 2017.
(60) Provisional application No. 62/472,519, filed on Mar. 16, 2017.
(60) Provisional application No. 62/444,860, filed on Jan. 11, 2017.
(60) Provisional application No. 61/897,245, filed on Oct. 30, 2013.
(60) Provisional application No. 61/126,047, filed on May 1, 2008.
(60) Provisional application No. 61/126,027, filed on May 1, 2008.

(51) Int. Cl. A61B 17/12

(52) U.S. Cl. CPC A61B 17/12113; A61B 17/1215

(57) ABSTRACT

This invention is an intrasaccular aneurysm occlusion device with a flexible net or mesh which is deployed into an aneurysm sac and then expanded by the insertion of polymer longitudinal embolic members (e.g. polymer tubular strands) into the net or mesh. As the net or mesh expands, it comes into contact with the walls of the aneurysm sac and conforms to the shape of even an irregularly-shaped aneurysm sac.

### CROSS-REFERENCE TO RELATED APPLICATIONS

This present application is a continuation-in-part of patent application Ser. No. 17/220,002 filed on 2021 Apr. 1. This present application is also a continuation-in-part of patent application Ser. No. 17/214,827 filed on 2021 Mar. 27. This present application is also a continuation-in-part of patent application Ser. No. 17/211,446 filed on 2021 Mar. 24. This present application claims the priority benefit of provisional patent application 63/119,774 filed on 2020 Dec. 1. This present application is also a continuation-in-part of patent application Ser. No. 16/693,267 filed on 2019 Nov. 23. This present application is also a continuation-in-part of patent application Ser. No. 16/660,929 filed on 2019 Oct. 23.

Application Ser. No. 16/693,267 is a continuation-in-part of patent application Ser. No. 16/660,929 filed on 2019 Oct. 23. Application Ser. No. 16/693,267 claimed the priority benefit of provisional patent application 62/794,609 filed on 2019 Jan. 19. Application Ser. No. 16/693,267 claimed the priority benefit of provisional patent application 62/794,607 filed on 2019 Jan. 19. Application Ser. No. 16/693,267 was a continuation-in-part of patent application Ser. No. 16/541,241 filed on 2019 Aug. 15. Application Ser. No. 16/693,267 was a continuation-in-part of patent application Ser. No. 15/865,822 filed on 2018 Jan. 9 and issued as patent Ser. No. 10/716,573 on 2020 Jul. 21. Application Ser. No. 16/693,267 was a continuation-in-part of patent application Ser. No. 15/861,482 filed on 2018 Jan. 3.

Application Ser. No. 16/660,929 claimed the priority benefit of provisional patent application 62/794,609 filed on 2019 Jan. 19. Application Ser. No. 16/660,929 claimed the priority benefit of provisional patent application 62/794,607 filed on 2019 Jan. 19. Application Ser. No. 16/660,929 was a continuation-in-part of patent application Ser. No. Ser. No. 16/541,241 filed on 2019 Aug. 15. Application Ser. No. 16/660,929 was a continuation-in-part of patent application Ser. No. 15/865,822 filed on 2018 Jan. 9 and issued as patent Ser. No. 10/716,573 on 2020 Jul. 21. Application Ser. No. 16/660,929 was a continuation-in-part of patent application Ser. No. 15/861,482 filed on 2018 Jan. 3.

Application Ser. No. 16/541,241 claimed the priority benefit of provisional patent application 62/794,609 filed on 2019 Jan. 19. Application Ser. No. 16/541,241 claimed the priority benefit of provisional patent application 62/794,607 filed on 2019 Jan. 19. Application Ser. No. 16/541,241 claimed the priority benefit of provisional patent application 62/720,173 filed on 2018 Aug. 21. Application Ser. No. 16/541,241 was a continuation-in-part of patent application Ser. No. 15/865,822 filed on 2018 Jan. 9 and issued as patent Ser. No. 10/716,573 on 2020 Jul. 21.

Application Ser. No. 15/865,822 claimed the priority benefit of provisional patent application 62/589,754 filed on 2017 Nov. 22. Application Ser. No. 15/865,822 claimed the priority benefit of provisional patent application 62/472,519 filed on 2017 Mar. 16. Application Ser. No. 15/865,822 was a continuation-in-part of patent application Ser. No. 15/081,909 filed on 2016 Mar. 27. Application Ser. No. 15/865,822 was a continuation-in-part of patent application Ser. No. 14/526,600 filed on 2014 Oct. 29.

Application Ser. No. 15/861,482 claimed the priority benefit of provisional patent application 62/589,754 filed on 2017 Nov. 22. Application Ser. No. 15/861,482 claimed the priority benefit of provisional patent application 62/472,519 filed on 2017 Mar. 16. Application Ser. No. 15/861,482 claimed the priority benefit of provisional patent application 62/444,860 filed on 2017 Jan. 11. Application Ser. No. 15/861,482 was a continuation-in-part of patent application Ser. No. 15/080,915 filed on 2016 Mar. 25 and issued as patent Ser. No. 10/028,747 on 2018 Jul. 24. Application Ser. No. 15/861,482 was a continuation-in-part of patent application Ser. No. 14/526,600 filed on 2014 Oct. 29.

Application Ser. No. 15/081,909 was a continuation-in-part of patent application Ser. No. 14/526,600 filed on 2014 Oct. 29. Application Ser. No. 15/080,915 was a continuation-in-part of patent application Ser. No. 14/526,600 filed on 2014 Oct. 29. Application Ser. No. 14/526,600 claimed the priority benefit of provisional patent application 61/897,245 filed on 2013 Oct. 30. Application Ser. No. 14/526,600 was a continuation-in-part of patent application Ser. No. 12/989,048 filed on 2010 Oct. 21 and issued as U.S. Pat. No. 8,974,487 on 2015 Mar. 10. Application Ser. No. 12/989,048 claimed the priority benefit of provisional patent application 61/126,047 filed on 2008 May 1. Application Ser. No. 12/989,048 claimed the priority benefit of provisional patent application 61/126,027 filed on 2008 May 1.

The entire contents of these related applications are incorporated herein by reference.

FEDERALLY SPONSORED RESEARCH:

Not Applicable

### SEQUENCE LISTING OR PROGRAM

Not Applicable

BACKGROUND

### Field of Invention

This invention relates to devices for occluding cerebral aneurysms.

### Introduction

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain. Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Sadly, even among those who survive, approximately one-half suffer significant and permanent deterioration of brain function. Better alternatives for cerebral aneurysm treatment are needed.

### REVIEW OF THE RELEVANT ART

U.S. Pat. No. 6,312,421 (Boock, Nov. 6, 2001, “Aneurysm Embolization Material and Device”) discloses treating an aneurysm with a biocompatible polymeric string. U.S. patent application 20080319522 (von Lehe et al., Dec. 25, 2008, “Aneurysm Filler Detacher”) discloses an aneurysm filler detacher mechanism. U.S. patent application 20080319523 (Young, Dec. 25, 2008, “Aneurysm Filler Device”) discloses an aneurysm filler device comprising a pusher portion, a filler portion, and a bridge portion. U.S. patent application 20170215887 (Ferry, Aug. 3, 2017, “Method for Increasing Packing Density of Embolization Material and Detaching Embolization Material”) discloses embolization material comprising a tubular strand. U.S. Pat. No. 9,731,045 (Gall et al., Aug. 15, 2017, “Shape Memory Polymer”) discloses a polymer made from a linear chain acrylate and a multi-functional acrylate cross-linker. U.S. Pat. No. 9,808,252 (Trommeter et al., Nov. 17, 2017, “Vascular Occlusion Devices”) discloses Shape Memory Materials (SMM) for coil-shaped vascular occlusion devices. U.S. patent Ser. No. 10/201,351 (Castleberry et al., Feb. 12, 2019, “Continuous Embolic Coil and Methods and Devices for Delivery of the Same”) discloses a trimmable continuous embolic coil that is cut to length at a target occlusion site.

U.S. patent application 20150196744 (Aboytes, Jul. 16, 2015, “Devices and Method for Vascular Recanalization”) and U.S. Pat. No. 9,931,495 (Aboytes, Apr. 3, 2018, “Devices and Methods for Vascular Recanalization”) disclose a device for restoring blood flow through an obstructed blood vessel. U.S. Pat. No. 8,974,512 (Aboytes et al., Mar. 10, 2015, “Devices and Methods for the Treatment of Vascular Defects”), U.S. Pat. No. 8,998,947 (Aboytes et al., Apr. 7, 2015, “Devices and Methods for the Treatment of Vascular Defects”), and U.S. Pat. No. 9,844,382 (Aboytes et al., Dec. 19, 2017, “Devices and Methods for the Treatment of Vascular Defects”), and U.S. patent applications 20120239074 (Aboytes et al., Sep. 20, 2012, “Devices and Methods for the Treatment of Vascular Defects”), 20130116722 (Aboytes et al., May 9, 2013, “Devices and Methods for the Treatment of Vascular Defects”), 20150209050 (Aboytes et al., Jul. 30, 2015, “Devices and Methods for the Treatment of Vascular Defects”), 20150272590 (Aboytes et al., Oct. 1, 2015, “Devices and Methods for the Treatment of Vascular Defects”), 20150342613 (Aboytes et al., Dec. 3, 2015, “Devices and Methods for the Treatment of Vascular Defects”), 20160262766 (Aboytes et al., Sep. 15, 2016, “Devices and Methods for the Treatment of Vascular Defects”), 20180125501 (Aboytes et al., May 10, 2018, “Devices and Methods for the Treatment of Vascular Defects”), 20180132859 (Aboytes et al., May 17, 2018, “Devices and Methods for the Treatment of Vascular Defects”), 20180132862 (Aboytes et al., May 17, 2018, “Devices and Methods for the Treatment of Vascular Defects”), 20190105054 (Aboytes et al., Apr. 11, 2019, “Devices and Methods for the Treatment of Vascular Defects”), 20190105056 (Aboytes et al., Apr. 11, 2019, “Devices and Methods for the Treatment of Vascular Defects”), and 20180036012 (Aboytes et al., Feb. 8, 2018, “Devices, Systems, and Methods for the Treatment of Vascular Defects”) disclose intrasaccular ribbons for aneurysm occlusion. U.S. patent Ser. No. 10/617,426 (Aboytes et al., Apr. 14, 2020, “Devices and Methods for the Treatment of Vascular Defects”), Ser. No. 10/617,427 (Aboytes et al., Apr. 14, 2020, “Devices and Methods for the Treatment of Vascular Defects”), and Ser. No. 10/675,037 (Aboytes et al., Jun. 9, 2020, “Devices and Methods for the Treatment of Vascular Defects”), as well as U.S. patent application 20200205841 (Aboytes et al., Jul. 2, 2020, “Devices, Systems, and Methods for the Treatment of Vascular Defects”), disclose a ribbon-like intrasacular implant with first and second portions, wherein the first and second portions have a first configuration in which are linearly aligned and a second configuration in which they overlap.

U.S. patent application 20020026210 (Abdel-Gawwad, Feb. 28, 2002, “Endovascular Aneurysm Treatment Device and Method”) discloses using an intrasacular frame and suction to collapse an aneurysm. U.S. patent application 20190307546 (Aguilar et al., Oct. 10, 2019, “Embolic Device with Improved Neck Coverage”) discloses a helical intrasaccular device. U.S. patent application 20190223883 (Anderson et al., Jul. 25, 2019, “Occlusive Medical Device with Delivery System”) discloses a neck bridge to occlude a heart appendage. U.S. patent application 20190251866 (Babiker et al., Aug. 15, 2019, “Device Specific Finite Element Models for Simulating Endovascular Treatment”) discloses using finite element medical device models and computational fluid dynamics for aneurysm treatment. U.S. patent Ser. No. 10/426,487 (Bachman et al., Oct. 1, 2019, “Devices, Systems and Methods for Enclosing an Anatomical Opening”) discloses a device with a distal-facing portion which occludes an aneurysm and a proximal-facing portion which arches over lumina of an artery.

U.S. Pat. No. 9,980,733 (Badruddin et al., May 29, 2018, “System for and Method of Treating Aneurysms”) and Ser. No. 10/856,879 (Badruddin et al., Dec. 8, 2020, “System for and Method of Treating Aneurysms”) disclose an occlusion device with a cover and an inner anchoring member, wherein the cover expands to a diameter greater than an aneurysm neck and the inner anchoring member contacts the interior of the aneurysm. U.S. patent application 20200367895 (Badruddin et al., Nov. 26, 2020, “Systems and Methods for Treating Aneurysms”) and patent Ser. No. 10/856,880 (Badruddin et al., Dec. 8, 2020, “Systems and Methods for Treating Aneurysms”) disclose an implantable device with a proximal end seated against an aneurysm adjacent the neck and a distal end extending in the sac away from the neck.

U.S. patent application 20020026217 (Baker et al., Feb. 28, 2002, “Apparatus and Method for Repair of Perigraft Flow”) discloses a device for causing thrombus between a graft and an aneurysm wall. U.S. patent applications 20170079661 (Bardsley et al., Mar. 23, 2017, “Occlusive Devices”) and 20190269411 (Bardsley et al., Sep. 5, 2019, “Occlusive Devices”) disclose dual-layer inverted meshes for vascular occlusion. U.S. patent Ser. No. 10/314,593 (Bardsley et al., Jun. 11, 2019, “Occlusive Devices”) discloses dual-layer inverted meshes for vascular occlusion. U.S. patent application 20060052816 (Bates et al., Mar. 9, 2006, “Device for Treating an Aneurysm”) discloses a patch that covers an aneurysm neck. U.S. Pat. No. 9,393,022 (Becking et al., Jul. 19, 2016, “Two-Stage Deployment Aneurysm Embolization Devices”) discloses embolic implants which are deployed in two stages. U.S. patent application 20170252190 (Becking et al., Sep. 7, 2017, “Braid Implant Delivery Systems”) discloses neurovascular devices with low profile compressibility. U.S. patent application 20200367904 (Becking et al., Nov. 26, 2020, “Multiple Layer Filamentary Devices for Treatment of Vascular Defects”) discloses braided balls which reduce blood flow into an aneurysm. U.S. patent application 20190262002 (Benjamin, Aug. 29, 2019, “Novel Enhanced Orb-Like Intrasacular Device”) discloses an orb-shaped device with zones of flexure and open cells.

U.S. Pat. No. 5,690,666 (Berenstein et al., Nov. 25, 1997, “Ultrasoft Embolism Coils and Process for Using Them”) discloses ultrasoft embolism coils. U.S. Pat. No. 7,695,488 (Berenstein et al., Apr. 13, 2010, “Expandable Body Cavity Liner Device”) discloses an aneurysm liner with areas with different elasticities. U.S. patent application 20040010263 (Boucher et al., Jan. 15, 2004, “Expandable Preformed Structures for Deployment in Interior Body Regions”) discloses using a stylet to straighten an expandable structure during deployment into an interior body region. U.S. patent Ser. No. 10/406,010 (Bourang, Sep. 10, 2019, “Multi-Stent and Multi-Balloon Apparatus for Treating Bifurcations and Methods of Use”) discloses using two catheters and three stents to treat a bifurcated vessel. U.S. patent application 20190046210 (Bowman, Feb. 14, 2019, “Embolic Device with Shaped Wire”) discloses using a helical carrier to occlude an aneurysm.

U.S. patent application 20190069900 (Cam et al., Mar. 7, 2019, “Vascular Remodeling Device”) discloses a vascular remodeling device with a first section and a protruding section. U.S. patent application 20210000624 (Cam et al., Jan. 7, 2021, “Vascular Remodeling Device”) discloses a vascular remodeling device with a proximal section, an intermediate section, and a distal section, wherein the distal section is positioned in a vessel bifurcation. U.S. patent application 20190167270 (Chen, Jun. 6, 2019, “Vaso-Occlusive Devices with In-Situ Stiffening”) discloses a vaso-occlusive device that is constructed out of dissimilar metallic materials which cause galvanic corrosion. U.S. patent application 20200170647 (Chen et al., Jun. 4, 2020, “Vaso-Occlusive Device”) discloses a gold-platinum alloy vaso-occlusive structure which is implanted in an aneurysm sac. U.S. patent application 20190133795 (Choubey, May 9, 2019, “Meshes, Devices and Methods for Treating Vascular Defects”) discloses stents with a plurality of strut regions and a plurality of bridge regions. U.S. patent application 20190192322 (Choubey et al., Jun. 27, 2019, “Vascular Flow Diversion”) discloses a device with a plurality of connector sections extending circumferentially about the device. U.S. patent applications 20190269534 (Choubey, Sep. 5, 2019, “Thin Film Mesh Hybrid for Treating Vascular Defects”) and 20170273692 (Choubey, Sep. 28, 2017, “Thin Wall Constructions for Vascular Flow Diversion”) disclose stents with strut regions extending circumferentially about the expandable device.

U.S. patent application 20120283768 (Cox et al., Nov. 8, 2012, “Method and Apparatus for the Treatment of Large and Giant Vascular Defects”) discloses a plurality of self-expanding globular shells which are inserted into an aneurysm sac. U.S. patent application 20170245862 (Cox et al., Aug. 31, 2017, “Methods and Devices for Treatment of Vascular Defects”) discloses a method for inserting a self-expanding globular shell into an aneurysm sac. U.S. patent application 20140052233 (Cox et al., Feb. 20, 2014, “Methods and Devices for Treatment of Vascular Defects”) discloses a self-expanding globular shell which is inserted into an aneurysm sac. U.S. Pat. No. 6,511,468 (Cragg et al., Jan. 28, 2003, “Device and Method for Controlling Injection of Liquid Embolic Composition”) discloses a system to deliver liquid embolic material into an aneurysm. U.S. patent application 20190150932 (Cruise et al., May 23, 2019, “Embolization Device Constructed from Expansile Polymer”) discloses expandable polymer devices for aneurysm occlusion. U.S. patent application 20190240050 (Dawson et al., Aug. 8, 2019, “Vascular Expandable Devices”) discloses a tubular structure made with a plurality of braided metallic elements. U.S. patent applications 20190239895 (Dawson et al., Aug. 8, 2019, “Vascular Expandable Devices”) and 20190240049 (Dawson et al., Aug. 8, 2019, “Vascular Expandable Devices”) disclose a device with a generally tubular sidewall formed by braided strands.

U.S. patent application 20200289125 (Dholakia et al., Sep. 17, 2020, “Filamentary Devices Having a Flexible Joint for Treatment of Vascular Defects”) discloses a permeable implant with first and second permeable shells, wherein the first permeable shell has a proximal end with a concave or recessed section and the second permeable shell has a convex section that mates with the concave or recessed section. U.S. patent application 20170281194 (Divino et al., Oct. 5, 2017, “Embolic Medical Devices”) discloses intrasaccular ribbons for aneurysm occlusion. U.S. patent Ser. No. 10/433,853 (Divino et al., Oct. 8, 2019, “Embolic Medical Devices”) discloses an intrasaccular ribbon for aneurysm occlusion with a pre-insertion rolled configuration. U.S. patent Ser. No. 10/327,781 (Divino et al., Jun. 25, 2019, “Occlusive Devices”) and U.S. patent application 20140135812 (Divino et al., May 15, 2014, “Occlusive Devices”) disclose intrasaccular occlusion which are filled with liquid embolic material and expand to a pre-set shape. U.S. patent applications 20190282242 (Divino et al., Sep. 19, 2019, “Occlusive Devices”) and 20190290286 (Divino et al., Sep. 26, 2019, “Occlusive Devices”) disclose intrasaccular occlusion devices which are filled with liquid embolic material and expand to a pre-set shape. U.S. patent application 20190343532 (Divino et al., Nov. 14, 2019, “Occlusive Devices”) discloses an intrasaccular device which changes from a compressed configuration to an expanded configuration with a unique shape or porosity profile.

U.S. patent Ser. No. 10/342,548 (Duncan, Jul. 9, 2019, “Occlusion Devices and Methods of Their Manufacture and Use”) discloses a device with a lateral fringe on membranous material. U.S. patent application 20190362496 (Dutta et al., Nov. 28, 2019, “Isolation of Aneurysm and Parent Vessel in Volumetric Image Data”) discloses a framework for isolating an aneurysm and a parent vessel in volumetric image data. U.S. patent application 20150216684 (Enzmann et al., Aug. 6, 2015, “Dual Rotational Stent Apparatus and Method for Endovascular Treatment of Aneurysms”) discloses a coaxial stent system for aneurysm treatment. U.S. patent application 20190307460 (Ferrera et al., Oct. 10, 2019, “Intrasacular Occlusion Devices Methods Processes and Systems”) discloses flexible aneurysm embolization devices made from laser cut nitinol. U.S. patent application 20200155333 (Franano et al., May 21, 2020, “Ballstent Device and Methods of Use”) discloses a round, thin-walled, expandable metal structure made from gold, platinum, or silver. U.S. patent application 20200163784 (Franano et al., May 28, 2020, “Blockstent Device and Methods of Use”) discloses a compressed, cylindrical or oblong, thin-walled, expandable stent for occluding a blood vessel segment.

U.S. patent application 20190223880 (Gerberding et al., Jul. 25, 2019, “Systems and Methods for Supporting or Occluding a Physiological Opening or Cavity”) discloses a device with a distal-facing portion which occludes an aneurysm and a proximal-facing portion which arches over lumina of an artery. U.S. Pat. No. 5,334,210 (Gianturco, Aug. 2, 1994, “Vascular Occlusion Assembly”) discloses an occlusion bag with an expanded diamond shape and an elongated flexible filler member. U.S. patent Ser. No. 10/939,915 (Gorochow et al., Mar. 9, 2021, “Aneurysm Device and Delivery System”) discloses a braid for treating an aneurysm, wherein the braid has a distal end opposite a proximal end, and wherein translating the braid causes a delivery portion to expand and form a distal sack as well as invert into itself. U.S. patent Ser. No. 10/653,425 (Gorochow et al., May 19, 2020, “Layered Braided Aneurysm Treatment Device”) discloses a tubular braid that is implanted in two distinct implanted shapes and a compaction-resistant column spanning the height of an aneurysm. U.S. patent Ser. No. 10/905,431 (Gorochow, Feb. 2, 2021, “Spiral Delivery System for Embolic Braid”) discloses a braided implant with a spiral segment.

U.S. patent application 20190216467 (Goyal, Jul. 18, 2019, “Apparatus and Methods for Intravascular Treatment of Aneurysms”) discloses an aneurysm neck bridge deployed in the parent vessel of the aneurysm. U.S. patent application 20180070955 (Greene et al., Mar. 15, 2018, “Embolic Containment”) discloses systems to deliver liquid embolic material into an aneurysm. U.S. Pat. No. 6,346,117 (Greenhalgh, Feb. 12, 2002, “Bag for Use in the Intravascular Treatment of Saccular Aneurysms”) and U.S. Pat. No. 6,391,037 (Greenhalgh, May 21, 2002, “Bag for Use in the Intravascular Treatment of Saccular Aneurysms”) disclose a plurality of resilient filamentary members braided into a tubular sleeve with an opening to receive a clotting medium such as a platinum wire.

U.S. Pat. No. 9,592,363 (Griffin et al., Mar. 14, 2017, “Medical Device”) discloses a device with a shaft having an elongated inner member and an elongated tubular reinforcing member disposed over at least a portion of the inner member. U.S. patent Ser. No. 10/130,372 (Griffin, Nov. 20, 2018, “Occlusion Device”), and U.S. patent applications 20150313605 (Griffin, Nov. 5, 2015, “Occlusion Device”), 20170156734 (Griffin, Jun. 8, 2017, “Occlusion Device”), 20190053810 (Griffin, Feb. 21, 2019, “Occlusion Device”), 20190059909 (Griffin, Feb. 28, 2019, “Occlusion Device”) disclose an occlusive mesh with a circumferential fold line. U.S. patent application 20190269414 (Griffin, Sep. 5, 2019, “Occlusion Device”) discloses an intrasaccular occlusion device with a plurality of coaxial expandable carriages. U.S. patent Ser. No. 10/869,672 (Griffin, Dec. 22, 2020, “Occlusion Device”) discloses an occlusion device with a solid marker and a resilient mesh body attached within the marker. U.S. patent application 20200038035 (Griffin, Feb. 6, 2020, “Occlusion Device”) discloses an occlusion device with a solid marker and a low profile resilient mesh body attached to the distal end of the marker. U.S. patent Ser. No. 10/285,711 (Griffin, May 14, 2019, “Occlusion Device”) discloses an occlusion device with a continuous compressible mesh structure comprising axial mesh carriages configured end to end, wherein each end of each carriage is a pinch point in the continuous mesh structure.

U.S. patent Ser. No. 10/426,486 (Guo et al., Oct. 1, 2019, “Vaso-Occlusive Device Delivery System”) discloses a vaso-occlusive device delivery system with a heat-activated pusher. U.S. patent application 20190262119 (Gupta et al., Aug. 29, 2019, “Delivery Device for Use with an Embolic Material”) discloses an embolic material delivery assembly with an outer member having a lumen extending therein, a distal end region, and an inner member disposed within the lumen of the outer member. U.S. patent applications 20200187953 (Hamel et al., Jun. 18, 2020, “Devices, Systems, and Methods for the Treatment of Vascular Defects”), 20200187954 (Hamel et al., Jun. 18, 2020, “Devices, Systems, and Methods for the Treatment of Vascular Defects”), 20200197017 (Hamel et al., Jun. 25, 2020, “Devices, Systems, and Methods for the Treatment of Vascular Defects”), 20200197018 (Hamel et al., Jun. 25, 2020, “Devices, Systems, and Methods for the Treatment of Vascular Defects”), and 20200197020 (Hamel et al., Jun. 25, 2020, “Devices, Systems, and Methods for the Treatment of Vascular Defects”) disclose a mesh which is deployed into a predetermined shape wherein: (a) the mesh is curved along its width, (b) the mesh is curved along its length, and (c) the mesh has an undulating contour across at least a portion of one or both of its length or its width. U.S. patent applications 20190209146 (Hebert et al., Jul. 11, 2019, “Micrograft for the Treatment of Intracranial Aneurysms and Method for Use”), 20190231328 (Hebert et al., Aug. 1, 2019, “Micrograft for the Treatment of Intracranial Aneurysms and Method for Use”), and 20190261967 (Hebert et al., Aug. 29, 2019, “Micrograft for the Treatment of Intracranial Aneurysms and Method for Use”) disclose a micrograft with a series of peaks and valleys formed by crimping.

U.S. patent applications 20180206849 (Hewitt et al., Jul. 26, 2018, “Filamentary Devices for the Treatment of Vascular Defects”) and 20170095254 (Hewitt et al., Apr. 6, 2017, “Filamentary Devices for Treatment of Vascular Defects”) disclose a self-expanding globular shell which is inserted into an aneurysm sac. U.S. patent application 20190223881 (Hewitt et al., Jul. 25, 2019, “Devices for Therapeutic Vascular Procedures”) discloses a self-expanding globular shell which is inserted into an aneurysm sac, wherein some shell filaments extend beyond the distal end of the shell. U.S. Pat. No. 9,955,976 (Hewitt et al., May 1, 2018, “Filamentary Devices for Treatment of Vascular Defects”) discloses a self-expanding intrasaccular globular shell with areas with different size pores. U.S. patent application 20160249934 (Hewitt et al., Sep. 1, 2016, “Filamentary Devices for Treatment of Vascular Defects”) discloses occlusive meshes with variable mesh density. U.S. patent application 20160249935 (Hewitt et al., Sep. 1, 2016, “Devices for Therapeutic Vascular Procedures”) discloses an expandable cylindrical structure made of wires with a self-expanding permeable shell at the distal end of the cylindrical structure. U.S. patent application 20160367260 (Hewitt et al., Dec. 22, 2016, “Devices for Therapeutic Vascular Procedures”) discloses an expandable cylindrical structure made of wires and a self-expanding permeable shell at the distal end of the cylindrical structure. U.S. patent application 20170128077 (Hewitt et al., May 11, 2017, “Devices for Therapeutic Vascular Procedures”) discloses methods and devices for removing a thrombus. U.S. Pat. No. 9,492,174 (Hewitt et al., Nov. 15, 2016, “Filamentary Devices for Treatment of Vascular Defects”) and Ser. No. 10/813,645 (Hewitt et al., Oct. 27, 2020, “Filamentary Devices for Treatment of Vascular Defects”) disclose a resilient self-expanding permeable implant having a plurality of elongate filaments which are woven together. U.S. patent Ser. No. 10/939,914 (Hewitt et al., Mar. 9, 2021, “Filamentary Devices for the Treatment of Vascular Defects”) discloses permeable shells made of woven braided mesh with variable mesh density. U.S. Pat. No. 9,629,635 (Hewitt et al., Apr. 25, 2017, “Devices for Therapeutic Vascular Procedures”) discloses an expandable cylindrical structure made of wires and a self-expanding permeable shell located at the distal end of the cylindrical structure. U.S. Pat. No. 9,078,658 (Hewitt et al., Jul. 14, 2015, “Filamentary Devices for Treatment of Vascular Defects”) discloses a self-expanding resilient permeable shell having an expanded state with a globular and longitudinally shortened configuration relative to a radially constrained state, and a plurality of elongate filaments which are woven together, which define a cavity of the permeable shell. U.S. patent application 20200289126 (Hewitt et al., Sep. 17, 2020, “Filamentary Devices for Treatment of Vascular Defects”) discloses a permeable implant with a stiff proximal portion that is configured to sit at the neck of an aneurysm.

U.S. patent application 20010034531 (Ho et al., Oct. 25, 2001, “Bioactive Three Loop Coil”) discloses an occlusion subassembly comprising a base section and a lateral protrusion fixedly attached to the base section. U.S. patent application 20050142163 (Hunter et al., Jun. 30, 2005, “Medical Implants and Fibrosis-Inducing Agents”) discloses implants with fibrosis-inducing agents. U.S. patent application 20190247053 (Inouye, Aug. 15, 2019, “Occlusive Medical Device”) discloses a neck bridge to occlude a heart appendage. U.S. patent application 20190298380 (Inouye et al., Oct. 3, 2019, “Occlusive Medical Device with Fixation Members”) discloses a neck bridge to occlude a heart appendage. U.S. Pat. No. 6,530,934 (Jacobsen et al., Mar. 11, 2003, “Embolic Device Composed of a Linear Sequence of Miniature Beads”) discloses an embolic device comprising a sequence of flexibly interconnected miniature beads. U.S. Pat. No. 6,585,748 (Jeffree, Jul. 1, 2003, “Device for Treating Aneurysms”) discloses a permeable intrasaccular bag into which embolic coils are inserted.

U.S. patent Ser. No. 10/405,966 (Johnson, Sep. 10, 2019, “Implantable Intraluminal Device”) discloses intraluminal stent graft devices whose walls include compliant channels which allow for fluid communication. U.S. Pat. No. 9,157,174 (Kusleika, Oct. 13, 2015, “Vascular Device for Aneurysm Treatment and Providing Blood Flow into a Perforator Vessel”) and U.S. Pat. No. 9,561,122 (Kusleika, Feb. 7, 2017, “Vascular Device for Aneurysm Treatment and Providing Blood Flow into a Perforator Vessel”) disclose occlusion devices with heat-set strands. U.S. patent application 20190133794 (Kusleika, May 9, 2019, “Methods and Systems for Increasing a Density of a Region of a Vascular Device”) discloses a stent with elastic members and differential porosity. U.S. patent application 20150005807 (Lagodzki et al., Jan. 1, 2015, “Occlusion Device Including Bundle of Occlusion Wires Having Preformed Shapes”) discloses an occlusion device with shape memory wires which expand to a preformed shape. U.S. patent application 20190216468 (Larsen et al., Jul. 18, 2019, “Occlusive Medical Device”) discloses a neck bridge to occlude a heart appendage. U.S. patent application 20170354402 (Lee et al., Dec. 14, 2017, “Braided Medical Devices”) discloses a vaso-occlusive member with helically-wound filaments.

U.S. patent application 20090318948 (Linder et al., Dec. 24, 2009, “Device, System and Method for Aneurysm Embolization”) discloses dispensing embolic elements freely and randomly within an aneurysm cavity. U.S. patent Ser. No. 10/716,574 (Lorenzo et al., Jul. 21, 2020, “Aneurysm Device and Delivery Method”) discloses a self-expanding braid with an outer occlusive sack and a segment which inverts into the outer occlusive sack like a tube sock. U.S. patent Ser. No. 10/905,430 (Lorenzo et al., Feb. 2, 2021, “Aneurysm Device and Delivery System”) discloses a braid for treating an aneurysm with a radially-expandable segment which forms an outer occlusive sack and a second radially-expandable segment which forms an inner occlusive sack. U.S. patent application 20210007755 (Lorenzo et al., Jan. 14, 2021, “Intrasaccular Aneurysm Treatment Device With Varying Coatings”) discloses an intrasaccular implant with an anti-thrombogenic coating. U.S. patent application 20190262123 (Mangiardi, Aug. 29, 2019, “Device and Method for Management of Aneurism, Perforation and Other Vascular Abnormalities”) discloses a method for treating perforations, fistulas, ruptures, dehiscence and aneurysms.

U.S. patent application 20130245667 (Marchand et al., Sep. 19, 2013, “Filamentary Devices and Treatment of Vascular Defects”) discloses a self-expanding globular shell which is inserted into an aneurysm sac. U.S. patent application 20160249937 (Marchand et al., Sep. 1, 2016, “Multiple Layer Filamentary Devices for Treatment of Vascular Defects”) discloses a self-expanding multi-layer shell which is inserted into an aneurysm sac. U.S. patent application 20180000489 (Marchand et al., Jan. 4, 2018, “Filamentary Devices for Treatment of Vascular Defects”) discloses a self-expanding globular shell which is inserted into an aneurysm sac. U.S. Pat. No. 9,597,087 (Marchand et al., Mar. 21, 2017, “Filamentary Devices for Treatment of Vascular Defects”) discloses a permeable shell configured to occlude blood flow. U.S. patent Ser. No. 10/610,231 (Marchand et al., Apr. 7, 2020, “Filamentary Devices for Treatment of Vascular Defects”) discloses a self-expanding resilient permeable shell having a plurality of elongate resilient filaments with a woven structure. U.S. patent application 20200281603 (Marchand et al., Sep. 10, 2020, “Filamentary Devices for Treatment of Vascular Defects”) discloses a permeable shell with proximal ends of filaments are gathered by a proximal hub and the distal ends of each of the filaments are gathered by a distal hub.

U.S. patent application 20190254691 (Martin et al., Aug. 22, 2019, “Flexible Intravascular Treatment Devices and Associated Systems and Methods of Use”) discloses stents with a plurality of cells and a plurality of joints between adjacent cells. U.S. patent application 20210052278 (Mauger, Feb. 25, 2021, “Vascular Occlusion Devices Utilizing Thin Film Nitinol Foils”) discloses a deployable occlusion device for filling an aneurysm with a first end portion and a second end portion, wherein the first end portion is attached to a support structure and the second end portion of the mesh component is a free end. U.S. patent application 20190209181 (Mayer et al., Jul. 11, 2019, “Medical Device for Treating Vascular Malformations”) discloses a helical device with a coilable section and a docking section. U.S. patent Ser. No. 10/595,875 (Mayer et al., Mar. 24, 2020, “Device for Restricting Blood Flow to Aneurysms”) and U.S. patent application 20200163677 (Mayer et al., May 28, 2020, “Device for Restricting Blood Flow to Aneurysms”) disclose a non-occlusive blood-restricting device with a sequence of loops having a gradually decreasing diameter.

U.S. patent application 20180271540 (Merritt et al., Sep. 27, 2018, “Systems and Methods for Embolization of Body Structures”) discloses a self-expanding shell with lobes which is inserted into an aneurysm sac. U.S. patent Ser. No. 10/881,413 (Merritt et al., Jan. 5, 2021, “Systems and Methods for Embolization of Body Structures”) discloses a self-expanding permeable shell with a plurality of circumferentially-arrayed lobes. U.S. patent application 20090112249 (Miles et al., Apr. 30, 2009, “Medical Device for Modification of Left Atrial Appendage and Related Systems and Methods”) discloses collapsible and self-expanding devices to modify a left atrial appendage. U.S. patent application 20210007754 (Milhous et al., Jan. 14, 2021, “Filamentary Devices for Treatment of Vascular Defects”) discloses a permeable implant having a radially constrained state configured for delivery within a catheter lumen, an expanded state, and a plurality of elongate filaments that are woven together. U.S. Pat. No. 9,687,245 (Molaei et al., Jun. 27, 2017, “Occlusive Devices and Methods of Use”) discloses an occlusive device with a proximal end, a distal end, and a lumen extending between the proximal and distal ends, wherein proximal end has a self-expanding distal section and the distal section has a coil portion.

U.S. patent applications 20180263629 (Murphy et al., Sep. 20, 2018, “Vaso-Occlusive Device and Delivery Assembly”) and 20190254676 (Murphy et al., Aug. 22, 2019, “Vaso-Occlusive Device and Delivery Assembly”) disclose a vaso-occlusive treatment system with multi-layer wires. U.S. patent application 20160213380 (O'Brien, et al., Jul. 28, 2016, “Occlusion Device Having Spherical Secondary Shape and Mandrel for Forming Same”) discloses a sphere made from helical memory wire. U.S. patent application 20190083075 (Onushko et al., Mar. 21, 2019, “Occlusive Medical Device with Sealing Member”) disclose a neck bridge to occlude a heart appendage. U.S. patent application 20060149309 (Paul et al., Jul. 6, 2006, “Inverting Occlusion Devices, Methods, and Systems”) discloses inverted vascular occlusion devices. U.S. patent application 20200367900 (Pedroso et al., Nov. 26, 2020, “Layered Braided Aneurysm Treatment Device With Corrugations”) discloses an implant with an open end, a pinched end, and a predetermined shape.

U.S. patent application 20170258473 (Plaza et al., Sep. 14, 2017, “Systems and Methods for Delivery of Stents and Stent-Like Devices”) discloses a self-expanding tubular structure which is inserted into the parent vessel of an aneurysm. U.S. patent application 20190046209 (Plaza et al., Feb. 14, 2019, “Delivery and Detachment Systems and Methods for Vascular Implants”) discloses a system for delivering an implant device to a vascular site. U.S. patent applications 20060155323 (Porter et al., Jul. 13, 2006, “Intra-Aneurysm Devices”) and 20190298379 (Porter et al., Oct. 3, 2019, “Intra-Aneurysm Devices”) disclose an aneurysm occlusion device with an upper member in the dome and a lower member in the aneurysm neck. U.S. patent application 20210052279 (Porter et al., Feb. 25, 2021, “Intra-Aneurysm Devices”) discloses an aneurysm occlusion device with an upper member that sits against the dome of the aneurysm, a lower member that sits in the neck of the aneurysm, and a means of adjusting the overall dimensions of the device. U.S. Pat. No. 4,638,803 (Rand, Jan. 27, 1987, “Medical Apparatus for Inducing Scar Tissue Formation in a Body”) discloses a balloon coated with thrombosis-inducing material.

U.S. patent application 20200289124 (Rangwala et al., Sep. 17, 2020, “Filamentary Devices for Treatment of Vascular Defects”) discloses a permeable implant with a stiffer proximal portion that is configured to sit at the neck of an aneurysm, wherein the stiffer proximal portion may include coils, stiffening elements, or reinforcement elements. U.S. patent application 20200038032 (Rhee et al., Feb. 6, 2020, “Occlusive Devices”) discloses a frame and a mesh component coupled to the frame, wherein mesh component has a first porosity, and the frame has a second porosity. U.S. patent application 20190209178 (Richter et al., Jul. 11, 2019, “Aneurysm Closure Device”) discloses occlusion of an aneurysm neck using a device with a plurality of self-expanding arms. U.S. patent applications 20140330299 (Rosenbluth et al., Nov. 6, 2014, “Embolic Occlusion Device and Method”) discloses a self-expanding globular shell which is inserted into an aneurysm sac. U.S. patent application 20180303486 (Rosenbluth et al., Oct. 25, 2018, “Embolic Occlusion Device and Method”) discloses a self-expanding globular shell which is inserted into an aneurysm sac plus a coil which extends out from the distal end of the shell. U.S. patent application 20160045201 (Rosenbluth et al., Feb. 18, 2016, “Blood Flow Disruption Devices and Methods for the Treatment of Vascular Defects”) discloses a blood flow disruption device with a porous inner flow disruption element and a porous outer flow disruption element which coaxially surrounds the inner flow disruption element.

U.S. patent application 20090287294 (Rosqueta et al., Nov. 19, 2009, “Braid-Ball Embolic Devices”) discloses “Goodness, Gracious, Great balls of wire!”. U.S. patent application 20190059907 (Rosqueta et al., Feb. 28, 2019, “Devices, Systems, and Methods for the Treatment of Vascular Defects”) discloses intrasaccular ribbons for aneurysm occlusion. U.S. patent Ser. No. 10/675,036 (Rosqueta et al., Jun. 9, 2020, “Devices, Systems, and Methods for the Treatment of Vascular Defects”) and U.S. patent application 20200138447 (Rosqueta et al., May 7, 2020, “Devices, Systems, and Methods for the Treatment of Vascular Defects”) disclose an occlusive device that includes a first mesh having an expanded state in which it curves about a first axis to form a first band, and a second mesh having an expanded state in which it curves about a second axis different than the first axis to form a second band. U.S. Pat. No. 6,350,270 (Roue, Feb. 26, 2002, “Aneurysm Liner”) discloses an aneurysm liner with an extender inside the liner. U.S. patent application 20160022445 (Ruvalcaba et al., Jan. 28, 2016, “Occlusive Device”), patent application 20190343664 (Ruvalcaba et al., Nov. 14, 2019, “Occlusive Device”), and patent Ser. No. 10/736,758 (Ruvalcaba et al., Aug. 11, 2020, “Occlusive Device”) disclose an aneurysm embolization device with a body having a single, continuous piece of material that is shape set into a plurality of distinct structural components.

U.S. Pat. No. 6,855,153 (Saadat, Feb. 15, 2005, “Embolic Balloon”) and U.S. patent application 20020165572 (Saadat, Nov. 7, 2002, “Embolic Balloon”) disclose an embolic balloon which aspirates blood while expanding. U.S. patent application 20110184451 (Sahl, Jul. 28, 2011, “Membrane Implant for Treatment of Cerebral Artery Aneurysms”) discloses a cylindrical biocompatible plastic membrane used in combination with a stent. U.S. Pat. No. 5,041,090 (Scheglov et al., Aug. 20, 1991, “Occluding Device”) discloses using nested balloons for occlusion. U.S. patent application 20020169473 (Sepetka et al., Nov. 14, 2002, “Devices and Methods for Treating Vascular Malformations”) discloses occlusive devices with a primary coil and secondary windings. U.S. patent application 20080281350 (Sepetka et al., Nov. 13, 2008, “Aneurysm Occlusion Devices”) discloses an (hourglass-shaped) occlusive device with a biocompatible matrix. U.S. patent application 20060116709 (Sepetka et al., Jun. 1, 2006, “Aneurysm Treatment Devices and Methods”) discloses a device which expands within an aneurysm sac. U.S. Pat. No. 8,597,320 (Sepetka et al., Dec. 3, 2013, “Devices and Methods for Treating Vascular Malformations”) discloses an occlusive device with a proximal collar and a distal collar. U.S. patent application 20190274691 (Sepetka et al., Sep. 12, 2019, “Occlusive Device”) discloses a tubular braid that folds inward on itself for aneurysm occlusion.

U.S. patent Ser. No. 10/420,862 (Sharma et al., Sep. 24, 2019, “In-Situ Forming Foams for Treatment of Aneurysms”) and U.S. patent application 20120265287 (Sharma et al., Oct. 18, 2012, “In-Situ Forming Foams for Treatment of Aneurysms”) disclose the use of in-situ forming polymer foams to treat aneurysms. U.S. patent Ser. No. 10/729,447 (Shimizu et al., Aug. 4, 2020, “Devices for Vascular Occlusion”) discloses an occlusive device, occlusive device delivery system, method of using, and method of delivering an occlusive device. U.S. patent application 20040254625 (Stephens et al., Dec. 16, 2004, “Inflatable Implant”) discloses an implant that is inflated with filler materials. U.S. patent application 20190167272 (Stephens et al., Jun. 6, 2019, “Inflatable Implant”) discloses an implant with a low profile when introduced into the body and a larger profile when it is inflated with one or more filler materials. U.S. Pat. No. 4,364,392 (Strother et al., Dec. 21, 1982, “Detachable Balloon Catheter”) discloses a balloon into which a carrier liquid is pumped. U.S. patent application 20060167494 (Suddaby, Jul. 27, 2006, “Aneurysm Repair Method and Apparatus”) discloses disks pressing against inner and outer sides of an aneurysm neck. U.S. patent application 20190201592 (Takahashi et al., Jul. 4, 2019, “Devices and Methods for Aneurysm Treatment”) discloses ways to reduce susceptibility artifacts in MRA images.

U.S. patent application 20030028209 (Teoh et al., Feb. 6, 2003, “Expandable Body Cavity Liner Device”) discloses an aneurysm liner for treating aneurysms of various shapes and sizes. U.S. patent application 20040098027 (Teoh et al., May 20, 2004, “Expandable Body Cavity Liner Device”) discloses various aneurysm treatment devices ranging from ball stents to permeable liners. U.S. Pat. No. 7,153,323 (Teoh et al., Dec. 26, 2006, “Aneurysm Liner with Multi-Segment Extender”) discloses an aneurysm liner with extender segments inside the liner. U.S. patent application 20170354418 (Teoh et al., Dec. 14, 2017, “Vaso-Occlusive Device Delivery System”) discloses a vaso-occlusive device delivery system with a heat-activated pusher. U.S. Pat. No. 6,958,061 (Truckai et al., Oct. 25, 2005, “Microspheres with Sacrificial Coatings for Vaso-Occlusive Systems”) discloses using a fluid to deliver microspheres for vascular occlusion. U.S. Pat. No. 4,341,218 (U, Jul. 27, 1982, “Detachable Balloon Catheter”) discloses a balloon with a hollow cylinder fastened at the neck of the balloon. U.S. Pat. No. 5,935,148 (Villar et al., Aug. 10, 1999, “Detachable, Varying Flexibility, Aneurysm Neck Bridge”) and U.S. Pat. No. 6,063,104 (Villar et al., May 16, 2000, “Detachable, Varying Flexibility, Aneurysm Neck Bridge”) disclose an aneurysm neck bridge with varying flexibility. U.S. patent application 20190269533 (Vong et al., Sep. 5, 2019, “Stent and Stent Delivery Device”) discloses a stent made from a single woven nitinol wire.

U.S. patent application 20110196413 (Wallace et al., Aug. 11, 2011, “System and Method for Retaining Vaso-Occlusive Devices within an Aneurysm”) discloses an occlusive mesh made from a shape-memory alloy. U.S. patent applications 20170086851 (Wallace et al., Mar. 30, 2017, “Vaso-Occlusive Devices and Methods of Use”) and 20190201000 (Wallace et al., Jul. 4, 2019, “Vaso-Occlusive Devices”) disclose a vaso-occlusive delivery system with a pusher. U.S. patent Ser. No. 10/383,635 (Wallace et al., Aug. 20, 2019, “Vaso-Occlusive Devices and Methods of Use”) and U.S. patent application 20180250013 (Wallace et al., Sep. 6, 2018, “Vaso-Occlusive Devices Including a Friction Element”) disclose a vaso-occlusive system with a pusher to deliver soft embolic members. U.S. patent application 20200187952 (Walsh et al., Jun. 18, 2020, “Intrasaccular Flow Diverter for Treating Cerebral Aneurysms”) discloses flow diverters with a stabilizing frame for anchoring the implant and an occluding element for diverting blood flow from the aneurysm neck. U.S. patent application 20210022765 (Walzman, Jan. 28, 2021, “Coated Endovascular Intrasaccular Occlusion Device”) discloses an endovascular treatment mesh device with an amorphous hydrogel layer. U.S. patent Ser. No. 10/398,441 (Warner et al., Sep. 3, 2019, “Vascular Occlusion”) discloses an aneurysm occlusion system which includes a containment bag, a pusher, and a stopper ring. U.S. patent application 20030212419 (West, Nov. 13, 2003, “Aneurysm Embolization Device and Deployment System”) discloses an aneurysm embolization device with a headpiece and a plurality of spherical members.

U.S. Pat. No. 7,083,643 (Whalen et al., Aug. 1, 2006, “Methods for Treating Aneurysms”) discloses filling an aneurysm sac with a fluid composition which solidifies in situ. U.S. patent Ser. No. 10/898,199 (Wilson et al., Jan. 26, 2021, “Expandable Implant and Implant System”) discloses an expandable implant comprising a chain or linked sequence of expandable polymer foam elements. U.S. patent application 20200367897 (Wolfe et al., Nov. 26, 2020, “Systems and Methods for Treating Aneurysms”) discloses an inverted mesh tube having an outer layer and an inner layer, wherein the outer layer transitions to the inner layer at an inversion fold. U.S. patent application 20210045750 (Wolf et al., Feb. 18, 2021, “Systems and Methods for Treating Aneurysms”) discloses an implantable vaso-occlusive device including a proximal end configured to seat against the aneurysm adjacent the neck of the aneurysm and a distal end configured to extend in the sac and away from the neck of the aneurysm. U.S. patent application 20200367893 (Xu et al., Nov. 26, 2020, “Layered Braided Aneurysm Treatment Device”) discloses an implant with two layers of tubular braid set into a predetermined shape. U.S. patent application 20200367896 (Zaidat et al., Nov. 26, 2020, “Systems and Methods for Treating Aneurysms”) discloses an occlusion element with a first tubular mesh having a first end and a second end coupled together at a proximal end of the occlusion element, wherein an intermediate portion of the first tubular mesh includes a substantially 180 degree turn. U.S. patent Ser. No. 10/383,749 (Zhou et al., Aug. 20, 2019, “Stent and Method of Inserting a Stent into a Delivery Catheter”) discloses a stent which is radially contractable from a fully radially expanded state to a radially contracted state via elongation of the frame.

### SUMMARY OF THE INVENTION

This invention is an intrasaccular aneurysm occlusion device with a flexible net or mesh which is inserted into an aneurysm sac. After the flexible net or mesh has been inserted into the aneurysm sac, it is filled and expanded by the insertion of polymer longitudinal embolic members (e.g. polymer tubular strands) into the net or mesh. As the net or mesh expands, it comes into contact with the walls of the aneurysm sac and conforms to the shape of even an irregularly-shaped aneurysm sac. This is particularly useful for occluding non-spherical aneurysms without recanalization. In an example, the proximal portion of a net or mesh (closer to the aneurysm neck) can have a lower porosity and/or lower elasticity than the distal portion of the net or mesh (closer to the aneurysm dome). In an example, there can be a closeable opening in the proximal portion of the net or mesh through which the one or more polymer longitudinal embolic members are inserted into the net or mesh.

This device has the potential to occlude cerebral aneurysms more completely (higher fill volume and less recanalization), with fewer complications (no need for anti-platelet medications and less chance of device prolapse into the parent vessel), more rapidly (avoid time-consuming sequential placement of multiple types of coils), and at a lower cost (polymer can be less expensive than platinum) than aneurysm occlusion devices in the prior art.

### BRIEF INTRODUCTION TO THE FIGURES

FIGS. 1 through 4 show an aneurysm occlusion device with a flexible net or mesh which is filled and expanded by polymer longitudinal embolic members.

### DETAILED DESCRIPTION OF THE FIGURES

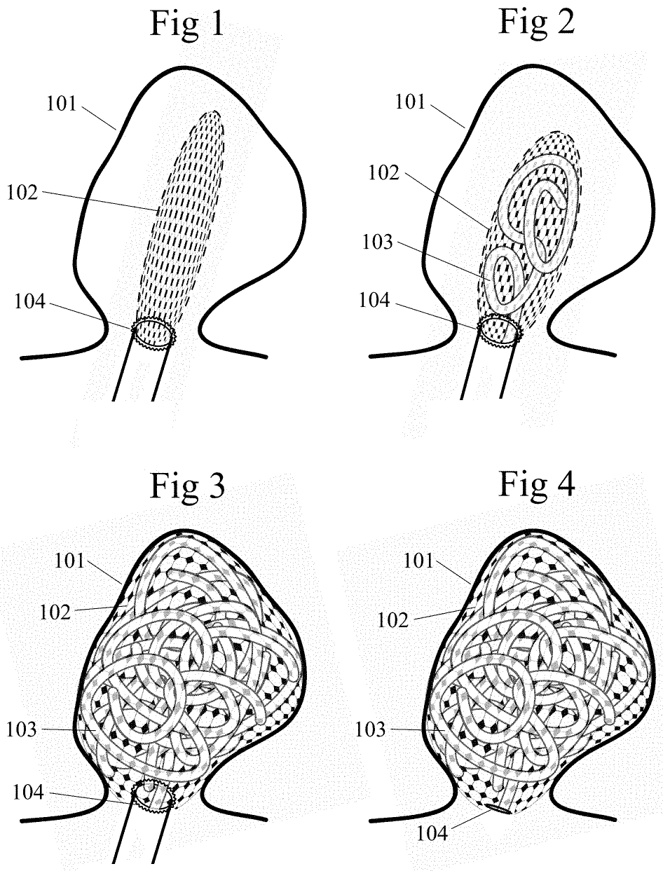

FIGS. 1 through 4 show four sequential views of an example of an intrasaccular device to occlude an aneurysm. FIG. 1 shows this device at a first point in time wherein the flexible net or mesh is inserted into the aneurysm sac. FIG. 2 shows this device at a second point in time as polymer longitudinal embolic members are being inserted into the flexible net or mesh, thereby expanding the flexible net or mesh. FIG. 3 shows this device at a third point in time after polymer longitudinal embolic members have expanded the flexible net or mesh to conform to the shape of the walls of the aneurysm sac and the closeable opening is being closed. FIG. 4 shows this device at a fourth point in time after the closeable opening has been closed and the device is fully deployed.

Specifically, FIGS. 1 through 4 show an intrasacular device to occlude an aneurysm comprising: a flexible net or mesh 102 which is configured to be inserted into an aneurysm sac 101, wherein there is a closeable proximal opening 104 on flexible net or mesh; and one or more polymer longitudinal embolic members 103 which are inserted through the closeable proximal opening into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the closeable proximal opening is closed after the one or more polymer longitudinal embolic members have been inserted into the net or mesh. Example variations discussed elsewhere in this disclosure or priority-linked disclosures can be incorporated into this example.

In an example, a device to occlude an aneurysm can comprise a flexible, expandable, and liquid-permeable net or mesh enclosure which is inserted into an aneurysm. This net or mesh enclosure can receive and retain a plurality of embolic fill members. Accumulation of the plurality of embolic fill members within the net or mesh enclosure causes the net or mesh enclosure to expand and to come into contact with and generally conform with an interior wall of the aneurysm. This can occlude the aneurysm and retain the net or mesh enclosure within the aneurysm.

In an example, a device to occlude an aneurysm can comprise: a plurality of embolic fill members; a conduit through which the embolic fill members are transported to an aneurysm sac; and a flexible, expandable, and liquid-permeable net or mesh enclosure, configured to be inserted into and to fit within the aneurysm sac, the net or mesh enclosure configured to receive and retain the plurality of embolic fill members such that a resulting accumulation of the plurality of embolic fill members within the net or mesh enclosure causes the net or mesh enclosure to expand and to come into contact with and generally conform with an interior wall of the aneurysm to thereby substantially occlude the aneurysm and retain the net or mesh enclosure within the aneurysm.

In an example, a method for occluding an aneurysm can comprise: inserting a flexible, expandable, and liquid-permeable net or mesh enclosure into an aneurysm; introducing a plurality of soft embolic fill members into the net or mesh enclosure; and retaining the fill members within the net or mesh enclosure such that a resulting accumulation of the plurality of fill members within the net or mesh enclosure causes the net or mesh enclosure to expand and to come into contact with and generally conform with an interior wall of the aneurysm to thereby substantially occlude the aneurysm and retain the net or mesh enclosure within the aneurysm.

In an example, a device to occlude an aneurysm can comprise: (a) a longitudinal lumen that is configured to be inserted into a blood vessel, wherein this blood vessel is the parent vessel from which an aneurysm has formed; (b) an expandable flexible net or mesh, wherein this expandable flexible net or mesh is configured to travel through the longitudinal lumen and to be inserted into the aneurysm sac, and wherein this net or mesh is sufficiently flexible to substantially conform to the walls of an irregularly shaped aneurysm sac after the net or mesh has been expanded; (c) a plurality of embolic members, wherein these embolic members are configured to travel through the longitudinal lumen and to be inserted into the net or mesh within the aneurysm sac; wherein these embolic members do not escape from the net or mesh; and wherein the net or mesh is expanded by the accumulation of embolic members inside the net or mesh.

In an example, a device to occlude an aneurysm can comprise: (a) a longitudinal lumen that is configured to be inserted into a blood vessel, wherein this blood vessel is the parent vessel from which an aneurysm has formed; (b) an expandable flexible net or mesh, wherein this expandable flexible net or mesh is configured to travel through the longitudinal lumen and to be inserted into the aneurysm sac, and wherein this net or mesh is sufficiently flexible to substantially conform to the walls of an irregularly shaped aneurysm sac after the net or mesh has been expanded; (c) a plurality of embolic members, wherein these embolic members are configured to travel through the longitudinal lumen and to be inserted into the net or mesh within the aneurysm sac; wherein these embolic members do not escape from the net or mesh; and wherein the net or mesh is expanded by the accumulation of embolic members inside the net or mesh; and (d) an expandable resilient structure, wherein this expandable resilient structure is configured to travel through the longitudinal lumen and to be inserted into the aneurysm sac; wherein this structure comes into engaging contact with the central circumference of the aneurysm sac when this structure is expanded; wherein this structure resists compression after it has been expanded; and wherein expansion of this structure also engages the net or mesh so as to prevent the net or mesh from slipping out from the aneurysm sac.

In an example, a device to occlude an aneurysm can comprise: (a) an intravascular delivery lumen; (b) a plurality of embolic members; (c) a neck bridging stent, wherein the neck bridging stent is configured to occlude the neck of an aneurysm sac; wherein the neck bridging stent has a first configuration while it is being transported through the delivery lumen to an aneurysm sac; wherein the neck bridging stent has a second configuration after it has left the delivery lumen and expanded within the aneurysm sac; wherein the neck bridging stent in its second configuration has a width in a plane which is substantially parallel to the circumference of the neck of the aneurysm sac and this width is greater than the diameter of the neck of the aneurysm sac; and wherein the neck bridging stent in its second configuration has a first level of flexibility, elasticity, and/or malleability; and (d) a net or mesh, wherein the net or mesh has a first configuration while it is being transported through the delivery lumen to an aneurysm sac; wherein the net or mesh has a second configuration after it has left the delivery lumen and been expanded within the aneurysm sac; wherein the net or mesh is configured to conform to the walls of an aneurysm sac in its second configuration; wherein the net or mesh is expanded from its first configuration to its second configuration by the insertion of the embolic members into the net or mesh; wherein the net or mesh in its second configuration has a second level of flexibility, elasticity, and/or malleability which is greater than the first level of flexibility, elasticity, and/or malleability.

In an example, a device to occlude an aneurysm can comprise: a net or mesh which is configured to be inserted into an aneurysm sac and expanded within the aneurysm sac; a stent which is configured to be inserted into the aneurysm sac and expanded within the aneurysm sac; a plurality of coils which are inserted into the net or mesh through a opening in the stent; and a closure mechanism which closes the opening in the stent. In an example, the net or mesh can be expanded by accumulation of coils in the net or mesh.

In an example, a device to occlude an aneurysm can comprise: a two-part mesh which is inserted into and expanded within an aneurysm sac, wherein the two-part mesh further comprises a bowl-shaped mesh and an outer net around the bowl-shaped mesh, wherein the bowl-shaped mesh has greater density, greater thickness, less porosity, and/or less flexibility than the net; and one or more longitudinal embolic members which are inserted into the net, wherein insertion of the embolic members into the net causes the net to flexibly expand and conform to the walls of the aneurysm sac.

In an example, embolic members which are inserted into a mesh or net can be flexible longitudinal embolic members. In an example, embolic members which are inserted into a mesh or net can be longitudinal embolic members. In an example, embolic members which are inserted into a mesh or net can be soft coils. In an example, embolic members which are inserted into a mesh or net can be hydrogels. In an example, embolic members which are inserted into a mesh or net can be hydrogels and/or coils. In an example, a net or mesh can be filled with embolic material such as hydrogels or coils.

In an example, embolic members which are inserted into a net or mesh can be made from a polymer. In an example, embolic members can include or consist essentially of one or more of the following, in isolation or in one or more polymers—cellulose, collagen, acetate, alginic acid, carboxy methyl cellulose, chitin, collagen glycosaminoglycan, divinylbenzene, ethylene glycol, ethylene glycol dimethylmathacrylate, ethylene vinyl acetate, hyaluronic acid, hydrocarbon polymer, hydroxyethylmethacrylate, methlymethacrylate, polyacrylic acid, polyamides, polyesters, polyolefins, polysaccharides, polyurethane, polyvinyl alcohol, silicone, urethane, and vinyl stearate.

In an example, embolic members can be made from one or more materials selected from the group consisting of: cellulose, collagen, acetate, alginic acid, carboxy methyl cellulose, chitin, collagen glycosaminoglycan, divinylbenzene, ethylene glycol, ethylene glycol dimethylmathacrylate, ethylene vinyl acetate, hyaluronic acid, hydrocarbon polymer, hydroxyethylmethacrylate, methlymethacrylate, polyacrylic acid, polyamides, polyesters, polyolefins, polysaccharides, polyurethane, polyvinyl alcohol, silicone, urethane, and vinyl stearate.

In an example, embolic members can be made from polyetherether ketone (PEEK). In an example, embolic members can be made from thermoplastic elastomer. In an example, embolic members can be made from polycarbonate urethane (PCU). In an example, embolic members can be made from water-soluble synthetic polymer. In an example, embolic members can be made from collagen. In an example, embolic members can be made from polyvinyl alcohol (PVA). In an example, embolic members can be made from polyether block amide (PEBA). In an example, embolic members can be made from radiopaque material. In an example, embolic members can be made from copolymer. In an example, embolic members can be made from polyvinyl pyrrolidone (PVP). In an example, embolic members can be made from polyesters. In an example, embolic members can be made from hydrogel.

In an example, a selected quantity, series, length, and/or volume of embolic members can be selectively dispensed and/or detached into the net or mesh in situ by a mechanism selected from the group consisting of: breaking a connection between embolic members in a series of embolic members; cutting a connection between embolic members in a series of embolic members (e.g. with a cutting edge or laser); dissolving a connection between embolic members in a series of embolic members (e.g. with thermal energy or a chemical); electrolytic mechanism; hydraulic mechanism; injecting a flow of embolic members suspended in a liquid or gel into a net or mesh; melting a connection between embolic members in a series of embolic members (e.g. with thermal or light energy); progressing embolic members into a net or mesh via a conveyor belt (e.g. chain-based conveyor); progressing embolic members into a net or mesh via a helical conveyor (e.g. with an Archimedes' screw); pushing embolic members into a net or mesh using the force of a liquid flow; pusher rod and/or plunger; release detachment mechanism; and thermal detachment mechanism.

In an example, a device to occlude an aneurysm can comprise a flexible, expandable, and liquid-permeable net or mesh which is inserted into an aneurysm. This net or mesh can receive and retain a plurality of embolic members. Accumulation of the plurality of embolic members within the net or mesh causes the net or mesh to expand and to come into contact with and generally conform with an interior wall of the aneurysm. This can occlude the aneurysm and retain the net or mesh within the aneurysm. In an example, a method for occluding an aneurysm can comprise: inserting a flexible, expandable, and liquid-permeable net or mesh into an aneurysm; introducing a plurality of soft embolic members into the net or mesh; and retaining the members within the net or mesh such that a resulting accumulation of the plurality of members within the net or mesh causes the net or mesh to expand and to come into contact with and generally conform with an interior wall of the aneurysm to thereby substantially occlude the aneurysm and retain the net or mesh within the aneurysm.

In an example, embolic members which are inserted into the mesh or net can be flexible longitudinal embolic fill members. In an example, these flexible longitudinal embolic fill members can be soft coils. In an example, a device to occlude an aneurysm can comprise: a plurality of longitudinal embolic members; a conduit through which the longitudinal embolic members are transported to an aneurysm sac; and a flexible, expandable, and liquid-permeable net or mesh, configured to be inserted into and to fit within the aneurysm sac, the net or mesh configured to receive and retain the plurality of longitudinal embolic members such that a resulting accumulation of the plurality of embolic members within the net or mesh enclosure causes the net or mesh to expand and to come into contact with and generally conform with an interior wall of the aneurysm to thereby substantially occlude the aneurysm and retain the net or mesh within the aneurysm.

In an example, flexible longitudinal embolic fill members which are inserted into a net or mesh can be made from a polymer. In an example, embolic fill members can include or consist essentially of one or more of the following, in isolation or in one or more polymers—cellulose, collagen, acetate, alginic acid, carboxy methyl cellulose, chitin, collagen glycosaminoglycan, divinylbenzene, ethylene glycol, ethylene glycol dimethylmathacrylate, ethylene vinyl acetate, hyaluronic acid, hydrocarbon polymer, hydroxyethylmethacrylate, methlymethacrylate, polyacrylic acid, polyamides, polyesters, polyolefins, polysaccharides, polyurethane, polyvinyl alcohol, silicone, urethane, and vinyl stearate.

In an example, a device to occlude an aneurysm can comprise: a plurality of longitudinal polymer embolic members; a conduit through which the longitudinal polymer embolic members are transported to an aneurysm sac; and a flexible, expandable, and liquid-permeable net or mesh, configured to be inserted into and to fit within the aneurysm sac, the net or mesh configured to receive and retain the plurality of longitudinal polymer embolic members such that a resulting accumulation of the plurality of embolic members within the net or mesh enclosure causes the net or mesh to expand and to come into contact with and generally conform with an interior wall of the aneurysm to thereby substantially occlude the aneurysm and retain the net or mesh within the aneurysm.

In an example, longitudinal embolic members which are inserted into a net or mesh can be made can be made from ethylene glycol. In an example, longitudinal embolic members which are inserted into a net or mesh can be made can be made from ethylene glycol dimethylmathacrylate. In an example, longitudinal embolic members which are inserted into a net or mesh can be made can be made from ethylene vinyl acetate. In an example, longitudinal embolic members can be made from PEEK (polyetherether ketone). In an example, longitudinal embolic members can be made from PVP (polyvinyl pyrrolidone). In an example, longitudinal embolic members can be made from PCU (polycarbonate urethane).

In an example, longitudinal embolic members which are inserted into a net or mesh can be made can be made from cellulose (e.g. cellulose acetate, cellulose acetate butyrate, cellulose acetate phthalate, carboxymethyl cellulose, cellulose ester, cellulose propionate, cellulose triacetate, ethyl cellulose, hydroxybutyl methyl cellulose, methyl cellulose, hydroxypropyl cellulose, and/or nitrocellulose). In an example, longitudinal embolic members which are inserted into a net or mesh can be made can be made from collagen. In an example, longitudinal embolic members which are inserted into a net or mesh can be made can be made from ethylene vinyl acetate and/or ethylene vinyl alcohol acetate. In an example, longitudinal embolic members which are inserted into a net or mesh can be made can be made from a hydrogel.

In an example, longitudinal embolic members which are inserted into a net or mesh can be made can be made from PEEK. In an example, longitudinal embolic members which are inserted into a net or mesh can be made can be made from PEG (polyethyleneglycol), PMMA (poly methyl methacrylate), and/or PEG-PMMA (polyethylene glycol-poly methyl methacrylate). In an example, longitudinal embolic members which are inserted into a net or mesh can be made can be made from PLGA (polyglycolic acid). In an example, longitudinal embolic members which are inserted into a net or mesh can be made from polyethylene (e.g., polyethylene glycol, polyethylene glycol dimethacrylate). In an example, longitudinal embolic members which are inserted into a net or mesh can be made can be made from polyvinyl (e.g. polyvinyl alcohol, polyvinyl chloride, polyvinyl ester, polyvinyl halide, polyvinyl pyrrolidone, polyvinyl acetate, polyvinyl phenol, polyvinyl pyrrolidone).

In an example, longitudinal embolic members can be made from a hydrogel. In an example, longitudinal embolic members which are inserted into a net or mesh can be made can be made from cellulose. In an example, longitudinal embolic members which are inserted into a net or mesh can be made can be made from carboxy methyl cellulose. In an example, longitudinal embolic members which are inserted into a net or mesh can be made can be made from chitin. In an example, longitudinal embolic members which are inserted into a net or mesh can be made can be made from collagen In an example, longitudinal embolic members which are inserted into a net or mesh can be made can be made from PVA (polyvinyl alcohol). In an example, longitudinal embolic members which are inserted into a mesh or net can be hydrogels. In an example, longitudinal embolic members which are inserted into a mesh or net can be hydrogels and/or coils. In an example, a net or mesh can be filled with embolic material such as hydrogels or coils.

In an example, a selected quantity, series, length, and/or volume of longitudinal embolic members can be selectively dispensed and/or detached into the net or mesh in situ by a mechanism selected from the group consisting of: breaking a connection between longitudinal embolic members in a series of longitudinal embolic members; cutting a connection between longitudinal embolic members in a series of longitudinal embolic members (e.g. with a cutting edge or laser); dissolving a connection between longitudinal embolic members in a series of longitudinal embolic members (e.g. with thermal energy or a chemical); electrolytic mechanism; hydraulic mechanism; injecting a flow of longitudinal embolic members suspended in a liquid or gel into a net or mesh; melting a connection between longitudinal embolic members in a series of longitudinal embolic members (e.g. with thermal or light energy); progressing longitudinal embolic members into a net or mesh via a conveyor belt (e.g. chain-based conveyor); progressing longitudinal embolic members into a net or mesh via a helical conveyor (e.g. with an Archimedes' screw); pushing longitudinal embolic members into a net or mesh using the force of a liquid flow; pusher rod and/or plunger; release detachment mechanism; and thermal detachment mechanism.

In an example, a device to occlude an aneurysm can comprise: an expandable flexible net or mesh, wherein this expandable flexible net or mesh is inserted into an aneurysm sac, and wherein this net or mesh is sufficiently flexible to substantially conform to the walls of an irregularly shaped aneurysm sac after the net or mesh has been expanded; and a plurality of longitudinal embolic members, wherein these longitudinal embolic members are configured to travel through the longitudinal lumen and to be inserted into the net or mesh within the aneurysm sac; wherein these longitudinal embolic members do not escape from the net or mesh; and wherein the net or mesh is expanded by the accumulation of longitudinal embolic members inside the net or mesh.

In an example, a device to occlude an aneurysm can comprise: (a) a longitudinal lumen that is configured to be inserted into a blood vessel, wherein this blood vessel is the parent vessel from which an aneurysm has formed; (b) an expandable flexible net or mesh, wherein this expandable flexible net or mesh is configured to travel through the longitudinal lumen and to be inserted into the aneurysm sac, and wherein this net or mesh is sufficiently flexible to substantially conform to the walls of an irregularly shaped aneurysm sac after the net or mesh has been expanded; (c) a plurality of longitudinal embolic members, wherein these longitudinal embolic members are configured to travel through the longitudinal lumen and to be inserted into the net or mesh within the aneurysm sac; wherein these longitudinal embolic members do not escape from the net or mesh; and wherein the net or mesh is expanded by the accumulation of longitudinal embolic members inside the net or mesh; and (d) an expandable resilient structure, wherein this expandable resilient structure is configured to travel through the longitudinal lumen and to be inserted into the aneurysm sac; wherein this structure comes into engaging contact with the central circumference of the aneurysm sac when this structure is expanded; wherein this structure resists compression after it has been expanded; and wherein expansion of this structure also engages the net or mesh so as to prevent the net or mesh from slipping out from the aneurysm sac.

In an example, a device to occlude an aneurysm can comprise: (a) an intravascular delivery lumen; (b) a plurality of longitudinal embolic members; (c) a neck bridging stent, wherein the neck bridging stent is configured to occlude the neck of an aneurysm sac; wherein the neck bridging stent has a first configuration while it is being transported through the delivery lumen to an aneurysm sac; wherein the neck bridging stent has a second configuration after it has left the delivery lumen and expanded within the aneurysm sac; wherein the neck bridging stent in its second configuration has a width in a plane which is substantially parallel to the circumference of the neck of the aneurysm sac and this width is greater than the diameter of the neck of the aneurysm sac; and wherein the neck bridging stent in its second configuration has a first level of flexibility, elasticity, and/or malleability; and (d) a net or mesh, wherein the net or mesh has a first configuration while it is being transported through the delivery lumen to an aneurysm sac; wherein the net or mesh has a second configuration after it has left the delivery lumen and been expanded within the aneurysm sac; wherein the net or mesh is configured to conform to the walls of an aneurysm sac in its second configuration; wherein the net or mesh is expanded from its first configuration to its second configuration by the insertion of the longitudinal embolic members into the net or mesh; wherein the net or mesh in its second configuration has a second level of flexibility, elasticity, and/or malleability which is greater than the first level of flexibility, elasticity, and/or malleability.

In an example, an intrasacular device to occlude an aneurysm can comprise: a polymer flexible net or mesh which is configured to be inserted into an aneurysm sac; a metal intrasacular neck-bridge which is configured to be inserted into the aneurysm sac and self-expand to span the interior of the aneurysm neck, wherein the neck-bridge is proximal (closer to the aneurysm neck) than the net or mesh; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac. In another example, a device to occlude an aneurysm can comprise: a net or mesh which is configured to be inserted into an aneurysm sac and expanded within the aneurysm sac; a stent which is configured to be inserted into the aneurysm sac and expanded within the aneurysm sac; a plurality of coils which are inserted into the net or mesh through a opening in the stent; and a closure mechanism which closes the opening in the stent. In another example, the net or mesh can be expanded by accumulation of coils in the net or mesh.

In an example, an intrasacular device to occlude an aneurysm can comprise: a flexible net or mesh which is configured to be inserted into an aneurysm sac; an intrasacular neck-bridge which is attached to the net or mesh and configured to be inserted into the aneurysm and self-expand to span the interior of the aneurysm neck, wherein the neck-bridge is proximal (closer to the aneurysm neck) than the net or mesh; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac.

In an example, an intrasacular device to occlude an aneurysm can comprise: a flexible net or mesh which is configured to be inserted into an aneurysm sac; an intrasacular neck-bridge which is configured to be inserted into the aneurysm sac outside the net or mesh and to self-expand to span the interior of the aneurysm neck, wherein the neck-bridge is proximal (closer to the aneurysm neck) than the net or mesh; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac.

Alternatively, an intrasacular device to occlude an aneurysm can comprise: a flexible net or mesh which is configured to be inserted into an aneurysm sac; an intrasacular neck-bridge which is configured to be inserted into the aneurysm sac inside the net or mesh and to self-expand to span the interior of the aneurysm neck, wherein the neck-bridge is proximal (closer to the aneurysm neck) than the net or mesh; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac. In another example, an intrasacular device to occlude an aneurysm can comprise: a flexible net or mesh which is configured to be inserted into an aneurysm sac; an intrasacular neck-bridge which is configured to be inserted into the aneurysm sac and self-expand to span the interior of the aneurysm neck, wherein the neck-bridge is proximal (closer to the aneurysm neck) than the net or mesh; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac.

In another possible embodiment, an intrasaccular device to occlude an aneurysm can comprise: a flexible and expandable net or mesh which is configured to be inserted into an aneurysm sac; and one or more longitudinal embolic members which are inserted into the net or mesh after the net or mesh has been inserted into the aneurysm sac, wherein the longitudinal embolic members further comprise a central wire and an outer polymer layer, and wherein accumulation of the embolic members in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac; and one or more longitudinal embolic members which are inserted into the net or mesh after the net or mesh has been inserted into the aneurysm sac, wherein the longitudinal embolic members further comprise a central wire and an outer hydrogel layer, and wherein accumulation of the embolic members in the net or mesh causes the net or mesh to expand, come into contact with the interior walls of the aneurysm sac, and conform to the shape of the interior walls of the aneurysm sac.

In an example, an intrasaccular device to occlude an aneurysm can comprise: a flexible and expandable net or mesh which is configured to be inserted into an aneurysm sac; and one or more longitudinal embolic members which are inserted into the net or mesh after the net or mesh has been inserted into the aneurysm sac, wherein the longitudinal embolic members further comprise a central wire and an outer expanding layer, and wherein accumulation of the embolic members in the net or mesh causes the net or mesh to expand, come into contact with the interior walls of the aneurysm sac, and conform to the shape of the interior walls of the aneurysm sac. In another example, a device to occlude an aneurysm can comprise: a two-part mesh which is inserted into and expanded within an aneurysm sac, wherein the two-part mesh further comprises a bowl-shaped mesh and an outer net around the bowl-shaped mesh, wherein the bowl-shaped mesh has greater density, greater thickness, less porosity, and/or less flexibility than the net; and one or more longitudinal embolic members which are inserted into the net, wherein insertion of the longitudinal embolic members into the net causes the net to flexibly expand and conform to the walls of the aneurysm sac.

Alternatively, an intrasaccular device to occlude an aneurysm can comprise: a flexible and expandable net or mesh which is configured to be inserted into an aneurysm sac, wherein a proximal portion of the net or mesh has a first level of porosity, a distal portion of the net or mesh has a second level of porosity, and the second level is greater than the first level; and one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac, wherein a proximal portion of the net or mesh has a first average pore size, a distal portion of the net or mesh has a second average pore size, and the second pore size is greater than the first pore size; and one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac.

In an example, an intrasaccular device to occlude an aneurysm can comprise: a flexible and expandable net or mesh which is configured to be inserted into an aneurysm sac, wherein a proximal portion of the net or mesh has a first level of flexibility, elasticity, and/or malleability, a distal portion of the net or mesh has a second level of flexibility, elasticity, and/or malleability, and the second level is greater than the first level; and one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac, wherein a proximal portion of the net or mesh has a first level of resiliency and/or radial strength, a distal portion of the net or mesh has a second level of resiliency and/or radial strength, and the second level is less than the first level; and one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac.

In another possible embodiment, an intrasaccular device to occlude an aneurysm can comprise: a flexible and expandable net or mesh which is configured to be inserted into an aneurysm sac, wherein a proximal portion of the net or mesh is composed of material with a first percentage of metal, up to 100%, a distal portion of the net or mesh is composed of material with a second percentage of metal, and the second percentage is less than the first percentage; and one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac, wherein a proximal portion of the net or mesh is composed of at least 75% metal, a distal portion of the net or mesh is composed of no more than 25% metal; and one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac.

Alternatively, an intrasaccular device to occlude an aneurysm can comprise: a flexible and expandable net or mesh which is configured to be inserted into an aneurysm sac, wherein a proximal portion of the net or mesh is composed of metal, a distal portion of the net or mesh is composed of a polymer; and one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac; and one or more polymer longitudinal embolic members which are inserted into the net or mesh after the net or mesh has been inserted into the aneurysm sac, wherein the embolic members are made from hydrogel, wherein accumulation of the embolic members in the net or mesh causes the net or mesh to expand, come into contact with the interior walls of the aneurysm sac, and conform to the shape of the interior walls of the aneurysm sac.

In an example, an intrasaccular device to occlude an aneurysm can comprise: a flexible and expandable net or mesh which is configured to be inserted into an aneurysm sac; and one or more polymer longitudinal embolic members which are inserted into the net or mesh after the net or mesh has been inserted into the aneurysm sac, wherein the embolic members are tubular polymer strands and/or soft polymer coils, and wherein accumulation of the embolic members in the net or mesh causes the net or mesh to expand, come into contact with the interior walls of the aneurysm sac, and conform to the shape of the interior walls of the aneurysm sac. In another example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac.

In an example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from PVP (polyvinyl pyrrolidone). In another example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from PEEK.

Alternatively, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from carboxy methyl cellulose. In another example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from PCU (polycarbonate urethane).

In another possible embodiment, an intrasacular device to occlude an aneurysm can comprise: a flexible net or mesh which is configured to be inserted into an aneurysm sac, wherein a proximal (closer to the aneurysm neck) portion of the net or mesh is made with a first percentage of metal (up to 100%), a distal (closer to the aneurysm dome) portion of the net or mesh is made with a second percentage of metal, and the second percentage is less than the first percentage; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac. In another example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from ethylene glycol.

In an example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from hydrogel. In another example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein a longitudinal segment of a polymer longitudinal embolic member is cut off (detached) after the segment has been inserted into the net or mesh.

Alternatively, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from PLGA (polyglycolic acid). In another example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein a longitudinal segment of a polymer longitudinal embolic member is cut off (detached) by a cutting edge after the segment has been inserted into the net or mesh.

In an example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein a longitudinal segment of a polymer longitudinal embolic member is cut off (detached) by a laser after the segment has been inserted into the net or mesh. In another example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein a longitudinal segment of a polymer longitudinal embolic member is cut off (detached) by thermal energy after the segment has been inserted into the net or mesh.

In another possible embodiment, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from polyvinyl (e.g. polyvinyl alcohol, polyvinyl chloride, polyvinyl ester, polyvinyl halide, polyvinyl pyrrolidone, polyvinyl acetate, polyvinyl phenol, polyvinyl pyrrolidone).

Alternatively, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from cellulose (e.g. cellulose acetate, cellulose acetate butyrate, cellulose acetate phthalate, carboxymethyl cellulose, cellulose ester, cellulose propionate, cellulose triacetate, ethyl cellulose, hydroxybutyl methyl cellulose, methyl cellulose, hydroxypropyl cellulose, and/or nitrocellulose).

In an example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from PVA (polyvinyl alcohol). In another example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from cellulose.

Alternatively, an intrasacular device to occlude an aneurysm can comprise: a flexible net or mesh which is configured to be inserted into an aneurysm sac, wherein a proximal (closer to the aneurysm neck) portion of the net or mesh has a first level of flexibility, elasticity, and/or malleability, a distal (closer to the aneurysm dome) portion of the net or mesh has a second level of flexibility, elasticity, and/or malleability, and the second level is greater than the first level; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac. In another example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from chitin.

In an example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from ethylene glycol dimethylmathacrylate. In another example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from PEEK (polyetherether ketone).

In an example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from ethylene vinyl acetate. In another example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from polyethylene (e.g., polyethylene glycol, polyethylene glycol dimethacrylate).

Alternatively, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from PEG (polyethyleneglycol), PMMA (poly methyl methacrylate), and/or PEG-PMMA (polyethylene glycol-poly methyl methacrylate).

In another possible embodiment, an intrasacular device to occlude an aneurysm can comprise: a flexible net or mesh which is configured to be inserted into an aneurysm sac, wherein a proximal (closer to the aneurysm neck) portion of the net or mesh has a first level of porosity, a distal (closer to the aneurysm dome) portion of the net or mesh has a second level of porosity, and the second level is greater than the first level; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac.

In an example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from collagen. In another example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein the one or more longitudinal embolic members are made from ethylene vinyl acetate and/or ethylene vinyl alcohol acetate.

In an example, an intrasacular device to occlude an aneurysm can comprise: a flexible net or mesh which is configured to be inserted into an aneurysm sac; and one or more polymer longitudinal embolic members which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac, and wherein a longitudinal segment of a polymer longitudinal embolic member is cut off (detached) by a hydraulic mechanism after the segment has been inserted into the net or mesh. In another example, an intrasacular device to occlude an aneurysm can comprise: a flexible net or mesh which is configured to be inserted into an aneurysm sac, wherein there is a closeable proximal opening in the flexible net or mesh; and one or more polymer longitudinal embolic members which are inserted through the proximal opening into the flexible and expandable net or mesh after the net or mesh has been inserted into the aneurysm sac; wherein accumulation of the one or more polymer longitudinal embolic members in the net or mesh causes the net or mesh to expand, come into contact with the interior walls of the aneurysm sac, and conform to the shape of the interior walls of the aneurysm sac; and wherein the proximal opening is closed after the one or more polymer longitudinal embolic members have been inserted into the net or mesh.

Alternatively, an intrasacular device to occlude an aneurysm can comprise: a flexible net or mesh which is configured to be inserted into an aneurysm sac, wherein there is a closeable proximal opening in the flexible net or mesh; and one or more polymer longitudinal embolic members which are inserted through the proximal opening into the flexible and expandable net or mesh after the net or mesh has been inserted into the aneurysm sac; wherein accumulation of the one or more polymer longitudinal embolic members in the net or mesh causes the net or mesh to expand, come into contact with the interior walls of the aneurysm sac, and conform to the shape of the interior walls of the aneurysm sac; and wherein the proximal opening is remotely closed by the device operator after the one or more polymer longitudinal embolic members have been inserted into the net or mesh. In another example, an intrasaccular device to occlude an aneurysm can comprise: a flexible and expandable net or mesh which is configured to be inserted into an aneurysm sac; and one or more polymer longitudinal embolic members which are inserted into the net or mesh after the net or mesh has been inserted into the aneurysm sac; wherein the embolic members are made from made from one or more materials selected from the group consisting of: cellulose acetate, cellulose acetate butyrate, cellulose acetate phthalate, carboxymethyl cellulose, cellulose ester, cellulose propionate, cellulose triacetate, ethyl cellulose, hydroxybutyl methyl cellulose, methyl cellulose, hydroxypropyl cellulose, and nitrocellulose; and wherein accumulation of the embolic members in the net or mesh causes the net or mesh to expand, come into contact with the interior walls of the aneurysm sac, and conform to the shape of the interior walls of the aneurysm sac.

In an example, an intrasaccular device to occlude an aneurysm can comprise: a flexible and expandable net or mesh which is configured to be inserted into an aneurysm sac; and one or more polymer longitudinal embolic members which are inserted into the net or mesh after the net or mesh has been inserted into the aneurysm sac; wherein the embolic members are made from one or more materials selected from the group consisting of: polyvinyl alcohol, polyvinyl chloride, polyvinyl ester, polyvinyl halide, polyvinyl pyrrolidone, polyvinyl acetate, polyvinyl phenol, and polyvinyl pyrrolidone; and wherein accumulation of the embolic members in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac; and one or more polymer longitudinal embolic members which are inserted into the net or mesh after the net or mesh has been inserted into the aneurysm sac; wherein the embolic members are made from one or more materials selected from the group consisting of: PEG (poly-ethylene-glycol), PMMA (poly-methyl-meth-acrylate), PLGA (poly-glycolic-acid), and PEEK (poly-ether-ether-ketone); and wherein accumulation of the embolic members in the net or mesh causes the net or mesh to expand, come into contact with the interior walls of the aneurysm sac, and conform to the shape of the interior walls of the aneurysm sac.

Alternatively, an intrasaccular device to occlude an aneurysm can comprise: a flexible and expandable net or mesh which is configured to be inserted into an aneurysm sac; and one or more polymer longitudinal embolic members which are inserted into the net or mesh after the net or mesh has been inserted into the aneurysm sac; wherein the embolic members are made from one or more materials selected from the group consisting of: chitin and collagen; and wherein accumulation of the embolic members in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac; and one or more polymer longitudinal embolic members which are inserted into the net or mesh after the net or mesh has been inserted into the aneurysm sac; wherein the embolic members have two concentric layers, an inner layer with a first durometer level and an outer layer with a second durometer level, wherein the second durometer level is lower than the first durometer level; and wherein accumulation of the embolic members in the net or mesh causes the net or mesh to expand, come into contact with the interior walls of the aneurysm sac, and conform to the shape of the interior walls of the aneurysm sac.

In an example, an intrasacular device to occlude an aneurysm can comprise: a flexible net or mesh which is configured to be inserted into an aneurysm sac; and one or more soft polymer coils which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac; and one or more soft polymer coils which are inserted into the net or mesh after the net or mesh has been inserted into the aneurysm sac, wherein accumulation of the soft polymer coils in the net or mesh causes the net or mesh to expand, come into contact with the interior walls of the aneurysm sac, and conform to the shape of the interior walls of the aneurysm sac.

In another possible embodiment, an intrasaccular device to occlude an aneurysm can comprise: a flexible and expandable net or mesh which is configured to be inserted into an aneurysm sac; and one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac, wherein a proximal portion of the net or mesh has a first level of porosity, a distal portion of the net or mesh has a second level of porosity, and the second level is greater than the first level; and one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac.

In an example, an intrasaccular device to occlude an aneurysm can comprise: a flexible and expandable net or mesh which is configured to be inserted into an aneurysm sac, wherein a proximal portion of the net or mesh has a first average pore size, a distal portion of the net or mesh has a second average pore size, and the second pore size is greater than the first pore size; and one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac, wherein a proximal portion of the net or mesh has a first level of flexibility, elasticity, and/or malleability, a distal portion of the net or mesh has a second level of flexibility, elasticity, and/or malleability, and the second level is greater than the first level; and one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac.

Alternatively, an intrasaccular device to occlude an aneurysm can comprise: a flexible and expandable net or mesh which is configured to be inserted into an aneurysm sac, wherein a proximal portion of the net or mesh has a first level of resiliency and/or radial strength, a distal portion of the net or mesh has a second level of resiliency and/or radial strength, and the second level is less than the first level; and one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac, wherein a proximal portion of the net or mesh is composed of material with a first percentage of metal, up to 100%, a distal portion of the net or mesh is composed of material with a second percentage of metal, and the second percentage is less than the first percentage; and one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac.

In an example, an intrasaccular device to occlude an aneurysm can comprise: a flexible and expandable net or mesh which is configured to be inserted into an aneurysm sac, wherein a proximal portion of the net or mesh is composed of at least 75% metal, a distal portion of the net or mesh is composed of no more than 25% metal; and one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac, wherein a proximal portion of the net or mesh is composed of metal, a distal portion of the net or mesh is composed of a polymer; and one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac.

In an example, an intrasaccular device to occlude an aneurysm can comprise: a flexible and expandable net or mesh which is configured to be inserted into an aneurysm sac; and one or more tubular polymer strands which are inserted into the net or mesh after the net or mesh has been inserted into the aneurysm sac, wherein the tubular polymer strands are made from hydrogel, wherein accumulation of the tubular polymer strands in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac; and one or more tubular polymer strands which are inserted into the net or mesh after the net or mesh has been inserted into the aneurysm sac, wherein the tubular polymer strands further comprise a central wire and an outer polymer layer, and wherein accumulation of the tubular polymer strands in the net or mesh causes the net or mesh to expand, come into contact with the interior walls of the aneurysm sac, and conform to the shape of the interior walls of the aneurysm sac.

In another possible embodiment, an intrasaccular device to occlude an aneurysm can comprise: a flexible and expandable net or mesh which is configured to be inserted into an aneurysm sac; and one or more tubular polymer strands which are inserted into the net or mesh after the net or mesh has been inserted into the aneurysm sac, wherein the tubular polymer strands further comprise a central wire and an outer hydrogel layer, and wherein accumulation of the tubular polymer strands in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac; and one or more tubular polymer strands which are inserted into the net or mesh after the net or mesh has been inserted into the aneurysm sac, wherein the tubular polymer strands further comprise a central wire and an outer expanding layer, and wherein accumulation of the tubular polymer strands in the net or mesh causes the net or mesh to expand, come into contact with the interior walls of the aneurysm sac, and conform to the shape of the interior walls of the aneurysm sac.

Alternatively, an intrasacular device to occlude an aneurysm can comprise: a flexible net or mesh which is configured to be inserted into an aneurysm sac; and one or more tubular polymer strands which are inserted into the flexible and expandable net or mesh after the net or mesh has been inserted into the aneurysm sac, wherein accumulation of the one or more polymer longitudinal embolic members in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac; and one or more tubular polymer strands which are inserted into the net or mesh after the net or mesh has been inserted into the aneurysm sac; wherein the tubular polymer strands are made from made from one or more materials selected from the group consisting of: cellulose acetate, cellulose acetate butyrate, cellulose acetate phthalate, carboxymethyl cellulose, cellulose ester, cellulose propionate, cellulose triacetate, ethyl cellulose, hydroxybutyl methyl cellulose, methyl cellulose, hydroxypropyl cellulose, and nitrocellulose; and wherein accumulation of the tubular polymer strands in the net or mesh causes the net or mesh to expand, come into contact with the interior walls of the aneurysm sac, and conform to the shape of the interior walls of the aneurysm sac.

In an example, an intrasaccular device to occlude an aneurysm can comprise: a flexible and expandable net or mesh which is configured to be inserted into an aneurysm sac; and one or more tubular polymer strands which are inserted into the net or mesh after the net or mesh has been inserted into the aneurysm sac; wherein the tubular polymer strands are made from one or more materials selected from the group consisting of: polyvinyl alcohol, polyvinyl chloride, polyvinyl ester, polyvinyl halide, polyvinyl pyrrolidone, polyvinyl acetate, polyvinyl phenol, and polyvinyl pyrrolidone; and wherein accumulation of the tubular polymer strands in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac; and one or more tubular polymer strands which are inserted into the net or mesh after the net or mesh has been inserted into the aneurysm sac; wherein the tubular polymer strands are made from one or more materials selected from the group consisting of: PEG (poly-ethylene-glycol), PMMA (poly-methyl-meth-acrylate), PLGA (poly-glycolic-acid), and PEEK (poly-ether-ether-ketone); and wherein accumulation of the tubular polymer strands in the net or mesh causes the net or mesh to expand, come into contact with the interior walls of the aneurysm sac, and conform to the shape of the interior walls of the aneurysm sac.

In an example, an intrasaccular device to occlude an aneurysm can comprise: a flexible and expandable net or mesh which is configured to be inserted into an aneurysm sac; and one or more tubular polymer strands which are inserted into the net or mesh after the net or mesh has been inserted into the aneurysm sac; wherein the tubular polymer strands are made from one or more materials selected from the group consisting of: chitin and collagen; and wherein accumulation of the tubular polymer strands in the net or mesh causes the net or mesh to expand, come into contact with the interior walls of the aneurysm sac, and conform to the shape of the interior walls of the aneurysm sac. In another example, an intrasaccular device to occlude an aneurysm can comprise: a flexible and expandable net or mesh which is configured to be inserted into an aneurysm sac; and one or more tubular polymer strands which are inserted into the net or mesh after the net or mesh has been inserted into the aneurysm sac; wherein the tubular polymer strands have two concentric layers, an inner layer with a first durometer level and an outer layer with a second durometer level, wherein the second durometer level is lower than the first durometer level; and wherein accumulation of the tubular polymer strands in the net or mesh causes the net or mesh to expand, come into contact with the interior walls of the aneurysm sac, and conform to the shape of the interior walls of the aneurysm sac.

In an example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a clipping mechanism, wherein the proximal end of a longitudinal segment of the polymer longitudinal embolic member is cut and detached by the clipping mechanism after the segment has been inserted into the net or mesh. In another example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a clipping mechanism, wherein the proximal end of a longitudinal segment of the polymer longitudinal embolic member is cut and detached by the clipping mechanism after the segment has been inserted into the net or mesh.

Alternatively, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a closeable opening in the center of the proximal end of the flexible net or mesh, wherein the longitudinal embolic members are inserted through the opening into the flexible and expandable net or mesh after the net or mesh has been inserted into the aneurysm sac, and wherein the opening is closed after the longitudinal embolic members have been inserted into the net or mesh. In another example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a closeable proximal opening in the flexible net or mesh, wherein the longitudinal embolic members are inserted through the proximal opening into the flexible and expandable net or mesh after the net or mesh has been inserted into the aneurysm sac, and wherein the proximal opening is closed after the longitudinal embolic members have been inserted into the net or mesh.

In an example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a closeable proximal opening in the net or mesh, wherein the longitudinal embolic members are inserted through the proximal opening into the net or mesh after the net or mesh has been inserted into the aneurysm sac, and wherein the proximal opening is closed by the device operator after the longitudinal embolic members have been inserted into the net or mesh.

In another possible embodiment, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a closeable proximal opening in the net or mesh, wherein the closeable opening, when open, has a diameter that is at least 25% greater than the cross-sectional diameter of the longitudinal embolic members, wherein the longitudinal embolic members are inserted through the opening into the net or mesh after the net or mesh has been inserted into the aneurysm sac, and wherein the opening is closed after the longitudinal embolic members have been inserted into the net or mesh.

In an example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a closeable proximal opening in the net or mesh, wherein the closeable opening, when open, has a diameter that is at least 50% greater than the cross-sectional diameter of the longitudinal embolic members, wherein the longitudinal embolic members are inserted through the opening into the net or mesh after the net or mesh has been inserted into the aneurysm sac, and wherein the opening is closed after the longitudinal embolic members have been inserted into the net or mesh. In another example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a cutting mechanism with a moving sharp edge, wherein a longitudinal segment of a polymer longitudinal embolic member is detached by the moving sharp edge after the segment has been inserted into the net or mesh.

Alternatively, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a cutting mechanism with a moving sharp edge, wherein a selected length of the polymer longitudinal embolic member is detached by the moving sharp edge after the segment has been inserted into the net or mesh. In another example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a cutting mechanism, wherein a longitudinal segment of a polymer longitudinal embolic member is detached by the cutting mechanism after the segment has been inserted into the net or mesh.

In an example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a cutting mechanism, wherein a selected length of the polymer longitudinal embolic member is detached by the cutting mechanism after the segment has been inserted into the net or mesh. In another example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a cutting mechanism, wherein the proximal end of a longitudinal segment of the polymer longitudinal embolic member is cut and detached by the cutting mechanism after the segment has been inserted into the net or mesh.

Alternatively, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a cutting mechanism, wherein the proximal end of a longitudinal segment of the polymer longitudinal embolic member is cut and detached by the cutting mechanism after the segment has been inserted into the net or mesh. In another example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a laser, wherein a longitudinal segment of a polymer longitudinal embolic member is detached by cutting by the laser after the segment has been inserted into the net or mesh.

In an example, an intrasaccular device to occlude an aneurysm can comprise: a polymer flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a metal intrasacular neck bridge which is configured to be inserted into the aneurysm sac and self-expand to span the interior of the aneurysm neck, wherein the neck-bridge is closer to the aneurysm neck than the net or mesh. In another example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a proximal closeable opening in the flexible net or mesh, wherein the longitudinal embolic members are inserted through the proximal opening into the flexible and expandable net or mesh after the net or mesh has been inserted into the aneurysm sac, and wherein the proximal opening is closed by thermal melding after the longitudinal embolic members have been inserted into the net or mesh.

In another possible embodiment, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a proximal opening with an elastic ring in the flexible net or mesh, wherein the longitudinal embolic members are inserted through the proximal opening into the flexible and expandable net or mesh after the net or mesh has been inserted into the aneurysm sac, and wherein the proximal opening is closed after the longitudinal embolic members have been inserted into the net or mesh.

In an example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a proximal opening with draw-string (or wire) closure mechanism in the flexible net or mesh, wherein the longitudinal embolic members are inserted through the proximal opening into the flexible and expandable net or mesh after the net or mesh has been inserted into the aneurysm sac, and wherein the proximal opening is closed by pulling the draw-string (or wire) after the longitudinal embolic members have been inserted into the net or mesh.

Alternatively, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a rotating cutting mechanism, wherein the proximal end of a longitudinal segment of the polymer longitudinal embolic member is cut and detached by rotation of the cutting mechanism after the segment has been inserted into the net or mesh. In another example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a rotating cutting mechanism, wherein the proximal end of a longitudinal segment of the polymer longitudinal embolic member is cut and detached by rotation of the cutting mechanism after the segment has been inserted into the net or mesh.

In an example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a sliding cutting mechanism, wherein the proximal end of a longitudinal segment of the polymer longitudinal embolic member is cut and detached by sliding of the cutting mechanism after the segment has been inserted into the net or mesh. In another example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and a sliding cutting mechanism, wherein the proximal end of a longitudinal segment of the polymer longitudinal embolic member is cut and detached by sliding of the cutting mechanism after the segment has been inserted into the net or mesh.

In an example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and an intrasacular neck bridge which is configured to be inserted into the aneurysm sac and self-expand to span the interior of the aneurysm neck, wherein the neck-bridge is closer to the aneurysm neck than the net or mesh. In another example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and an intrasacular neck bridge which is configured to be inserted into the aneurysm sac and self-expand to span the interior of the aneurysm neck, wherein the neck-bridge is closer to the aneurysm neck than the net or mesh.

Alternatively, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and an intrasacular neck bridge which is configured to be inserted into the aneurysm sac and self-expand to span the interior of the aneurysm neck, wherein the neck-bridge is closer to the aneurysm neck than the net or mesh, and wherein the intrasacular neck bridge is attached to the net or mesh. In another example, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and an intrasacular neck bridge which is configured to be inserted into the aneurysm sac and self-expand to span the interior of the aneurysm neck, wherein the neck-bridge is closer to the aneurysm neck than the net or mesh, and wherein the intrasacular neck bridge is inside the net or mesh.

In another possible embodiment, an intrasaccular device to occlude an aneurysm can comprise: a flexible and expandable net or mesh which is configured to be inserted into an aneurysm sac; one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac; and an intrasacular neck bridge which is configured to be inserted into the aneurysm sac and self-expand to span the interior of the aneurysm neck, wherein the neck-bridge is closer to the aneurysm neck than the net or mesh, and wherein the intrasacular neck bridge is outside the net or mesh. In another example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a clipping mechanism, wherein the proximal end of a longitudinal segment of the tubular polymer strand is cut and detached by the clipping mechanism after the segment has been inserted into the net or mesh.

Alternatively, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a clipping mechanism, wherein the proximal end of a longitudinal segment of the tubular polymer strand is cut and detached by the clipping mechanism after the segment has been inserted into the net or mesh. In another example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a closeable opening in the center of the proximal end of the flexible net or mesh, wherein the tubular polymer strands are inserted through the opening into the flexible and expandable net or mesh after the net or mesh has been inserted into the aneurysm sac, and wherein the opening is closed after the tubular polymer strands have been inserted into the net or mesh.

In an example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a closeable proximal opening in the flexible net or mesh, wherein the tubular polymer strands are inserted through the proximal opening into the flexible and expandable net or mesh after the net or mesh has been inserted into the aneurysm sac, and wherein the proximal opening is closed after the tubular polymer strands have been inserted into the net or mesh. In another example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a closeable proximal opening in the net or mesh, wherein the tubular polymer strands are inserted through the proximal opening into the net or mesh after the net or mesh has been inserted into the aneurysm sac, and wherein the proximal opening is closed by the device operator after the tubular polymer strands have been inserted into the net or mesh.

In an example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a closeable proximal opening in the net or mesh, wherein the closeable opening, when open, has a diameter that is at least 25% greater than the cross-sectional diameter of the tubular polymer strands, wherein the tubular polymer strands are inserted through the opening into the net or mesh after the net or mesh has been inserted into the aneurysm sac, and wherein the opening is closed after the tubular polymer strands have been inserted into the net or mesh.

In an example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a closeable proximal opening in the net or mesh, wherein the closeable opening, when open, has a diameter that is at least 50% greater than the cross-sectional diameter of the tubular polymer strands, wherein the tubular polymer strands are inserted through the opening into the net or mesh after the net or mesh has been inserted into the aneurysm sac, and wherein the opening is closed after the tubular polymer strands have been inserted into the net or mesh.

Alternatively, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a cutting mechanism with a moving sharp edge, wherein a longitudinal segment of a tubular polymer strand is detached by the moving sharp edge after the segment has been inserted into the net or mesh. In another example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a cutting mechanism with a moving sharp edge, wherein a selected length of the tubular polymer strand is detached by the moving sharp edge after the segment has been inserted into the net or mesh.

In another possible embodiment, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a cutting mechanism, wherein a longitudinal segment of a tubular polymer strand is detached by the cutting mechanism after the segment has been inserted into the net or mesh. In another example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a cutting mechanism, wherein a selected length of the tubular polymer strand is detached by the cutting mechanism after the segment has been inserted into the net or mesh.

Alternatively, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a cutting mechanism, wherein the proximal end of a longitudinal segment of the tubular polymer strand is cut and detached by the cutting mechanism after the segment has been inserted into the net or mesh. In another example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a cutting mechanism, wherein the proximal end of a longitudinal segment of the tubular polymer strand is cut and detached by the cutting mechanism after the segment has been inserted into the net or mesh.

In an example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a laser, wherein a longitudinal segment of a tubular polymer strand is detached by cutting by the laser after the segment has been inserted into the net or mesh. In another example, an intrasaccular device to occlude an aneurysm can comprise: a polymer flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a metal intrasacular neck bridge which is configured to be inserted into the aneurysm sac and self-expand to span the interior of the aneurysm neck, wherein the neck-bridge is closer to the aneurysm neck than the net or mesh.

In an example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a proximal closeable opening in the flexible net or mesh, wherein the tubular polymer strands are inserted through the proximal opening into the flexible and expandable net or mesh after the net or mesh has been inserted into the aneurysm sac, and wherein the proximal opening is closed by thermal melding after the tubular polymer strands have been inserted into the net or mesh.

In an example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a proximal opening with an elastic ring in the flexible net or mesh, wherein the tubular polymer strands are inserted through the proximal opening into the flexible and expandable net or mesh after the net or mesh has been inserted into the aneurysm sac, and wherein the proximal opening is closed after the tubular polymer strands have been inserted into the net or mesh.

Alternatively, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a proximal opening with draw-string (or wire) closure mechanism in the flexible net or mesh, wherein the tubular polymer strands are inserted through the proximal opening into the flexible and expandable net or mesh after the net or mesh has been inserted into the aneurysm sac, and wherein the proximal opening is closed by pulling the draw-string (or wire) after the tubular polymer strands have been inserted into the net or mesh. In another example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a rotating cutting mechanism, wherein the proximal end of a longitudinal segment of the tubular polymer strand is cut and detached by rotation of the cutting mechanism after the segment has been inserted into the net or mesh.

In an example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a rotating cutting mechanism, wherein the proximal end of a longitudinal segment of the tubular polymer strand is cut and detached by rotation of the cutting mechanism after the segment has been inserted into the net or mesh. In another example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a sliding cutting mechanism, wherein the proximal end of a longitudinal segment of the tubular polymer strand is cut and detached by sliding of the cutting mechanism after the segment has been inserted into the net or mesh.

Alternatively, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and a sliding cutting mechanism, wherein the proximal end of a longitudinal segment of the tubular polymer strand is cut and detached by sliding of the cutting mechanism after the segment has been inserted into the net or mesh. In another example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and an intrasacular neck bridge which is configured to be inserted into the aneurysm sac and self-expand to span the interior of the aneurysm neck, wherein the neck-bridge is closer to the aneurysm neck than the net or mesh.

In an example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and an intrasacular neck bridge which is configured to be inserted into the aneurysm sac and self-expand to span the interior of the aneurysm neck, wherein the neck-bridge is closer to the aneurysm neck than the net or mesh. In another example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and an intrasacular neck bridge which is configured to be inserted into the aneurysm sac and self-expand to span the interior of the aneurysm neck, wherein the neck-bridge is closer to the aneurysm neck than the net or mesh, and wherein the intrasacular neck bridge is attached to the net or mesh.

Alternatively, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and an intrasacular neck bridge which is configured to be inserted into the aneurysm sac and self-expand to span the interior of the aneurysm neck, wherein the neck-bridge is closer to the aneurysm neck than the net or mesh, and wherein the intrasacular neck bridge is inside the net or mesh.

In an example, an intrasaccular device to occlude an aneurysm can comprise: a flexible and expandable net or mesh which is configured to be inserted into an aneurysm sac; one or more tubular polymer strands which are inserted into the net or mesh after the net or mesh has been inserted into the aneurysm sac, wherein accumulation of the tubular polymer strands in the net or mesh causes the net or mesh to expand, come into contact with the interior walls of the aneurysm sac, and conform to the shape of the interior walls of the aneurysm sac; and an intrasacular neck bridge which is configured to be inserted into the aneurysm sac and self-expand to span the interior of the aneurysm neck, wherein the neck-bridge is closer to the aneurysm neck than the net or mesh, and wherein the intrasacular neck bridge is outside the net or mesh.

I claim:

1. An intrasacular device to occlude an aneurysm comprising:
a flexible and expandable net or mesh which is configured to be inserted into an aneurysm sac; and
one or more polymer longitudinal embolic members which are inserted into the net or mesh after the net or mesh has been inserted into the aneurysm sac, wherein accumulation of the embolic members in the net or mesh causes the net or mesh to expand, come into contact with the interior walls of the aneurysm sac, and conform to the shape of the interior walls of the aneurysm sac.

2. The device in claim 1 wherein the embolic members are tubular polymer strands and/or soft polymer coils.

3. The device in claim 1 wherein the embolic members are made from made from one or more materials selected from the group consisting of: cellulose acetate, cellulose acetate butyrate, cellulose acetate phthalate, carboxymethyl cellulose, cellulose ester, cellulose propionate, cellulose triacetate, ethyl cellulose, hydroxybutyl methyl cellulose, methyl cellulose, hydroxypropyl cellulose, and nitrocellulose.

4. The device in claim 1 wherein the embolic members are made from one or more materials selected from the group consisting of: polyvinyl alcohol, polyvinyl chloride, polyvinyl ester, polyvinyl halide, polyvinyl pyrrolidone, polyvinyl acetate, polyvinyl phenol, and polyvinyl pyrrolidone.

5. The device in claim 1 wherein the embolic members are made from one or more materials selected from the group consisting of: PEG (poly-ethylene-glycol), PMMA (poly-methyl-meth-acrylate), PLGA (poly-glycolic-acid), and PEEK (poly-ether-ether-ketone).

6. The device in claim 1 wherein the embolic members are made from one or more materials selected from the group consisting of: chitin and collagen.

7. The device in claim 1 wherein the embolic members are made from hydrogel.

8. The device in claim 1 wherein a longitudinal segment of a polymer longitudinal embolic member is detached by cutting after the segment has been inserted into the net or mesh.

9. The device in claim 1 wherein a longitudinal segment of a polymer longitudinal embolic member is detached by cutting by a moving sharp edge after the segment has been inserted into the net or mesh.

10. The device in claim 1 wherein a longitudinal segment of a polymer longitudinal embolic member is detached by cutting by a laser after the segment has been inserted into the net or mesh.

11. The device in claim 1 wherein there is a closeable proximal opening in the net or mesh, wherein the longitudinal embolic members are inserted through the proximal opening into the net or mesh after the net or mesh has been inserted into the aneurysm sac, and wherein the proximal opening is closed after the longitudinal embolic members have been inserted into the net or mesh.

12. The device in claim 1 wherein there is a closeable proximal opening in the net or mesh, wherein the longitudinal embolic members are inserted through the proximal opening into the net or mesh after the net or mesh has been inserted into the aneurysm sac, and wherein the proximal opening is closed by the device operator after the longitudinal embolic members have been inserted into the net or mesh.

13. The device in claim 1 wherein there is a closeable proximal opening in the net or mesh, wherein the closeable opening, when open, has a diameter that is at least 25% greater than the cross-sectional diameter of the longitudinal embolic members, wherein the longitudinal embolic members are inserted through the opening into the net or mesh after the net or mesh has been inserted into the aneurysm sac, and wherein the opening is closed after the longitudinal embolic members have been inserted into the net or mesh.

14. The device in claim 1 wherein a proximal portion of the net or mesh has a first level of porosity, a distal portion of the net or mesh has a second level of porosity, and the second level is greater than the first level.

15. The device in claim 1 wherein a proximal portion of the net or mesh has a first level of flexibility, elasticity, and/or malleability, a distal portion of the net or mesh has a second level of flexibility, elasticity, and/or malleability, and the second level is greater than the first level.

16. The device in claim 1 wherein a proximal portion of the net or mesh is composed of material with a first percentage of metal, up to 100%, a distal portion of the net or mesh is composed of material with a second percentage of metal, and the second percentage is less than the first percentage.

17. The device in claim 1 wherein the device further comprises an intrasacular neck bridge which is configured to be inserted into the aneurysm sac and self-expand to span the interior of the aneurysm neck, wherein the neck-bridge is closer to the aneurysm neck than the net or mesh.

18. The device in claim 17 wherein the intrasacular neck bridge is attached to the net or mesh.

19. The device in claim 17 wherein the intrasacular neck bridge is inside the net or mesh.

20. The device in claim 17 wherein the intrasacular neck bridge is outside the net or mesh.
